SEQUENCE LISTING (13 sequences)

SEQ ID NO: 1 - RNA, 27 nt, Artificial Sequence
  Description of Artificial Sequence Synthetic nucleotide sequence
cggaaucagugaaugcuuauacauccg

SEQ ID NO: 2 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVLSPRYEIVIITVVAAAVIMGTAGLSTYLYNRQRKIKKYRLQQAQKGTPMKPNTQATPP

SEQ ID NO: 3 - DNA, 2986 bp, Homo sapiens
gcgccccagtcgacgctgagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcgaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggcccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtccatgtcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttcacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactattgcagctccagtttcctgcagtgatcagggtcctgcaagcagtggggaagggggccaaggtattggaggactccctcccagctttggaagggtcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttttcagagacggggtctcgcaacattgcccagacttcctttgtgttagttaataaagctttctcaactgcc

SEQ ID NO: 4 - RNA, 10 nt, Artificial Sequence
  Description of Artificial Sequence Synthetic nucleotide sequence
gaagaauugg

SEQ ID NO: 5 - RNA, 8 nt, Artificial Sequence
  Description of Artificial Sequence Synthetic nucleotide sequence
uuggacgc

SEQ ID NO: 6 - RNA, 8 nt, Artificial Sequence
  Description of Artificial Sequence Synthetic nucleotide sequence
gugaaugc

SEQ ID NO: 7 - RNA, 27 nt, Artificial Sequence
  Description of Artificial Sequence Synthetic nucleotide sequence
cggaaucagugaaugcuuauacauccg

SEQ ID NO: 8 - RNA, 27 nt, Artificial Sequence
  Description of Artificial Sequence Synthetic nucleotide sequence
cggaaucagugaaugcuuauacauccg

SEQ ID NO: 9 - RNA, 27 nt, Artificial Sequence
  Description of Combined DNA/RNA Molecule Synthetic nucleotide sequence
cggaaucagugaaugcuuauacauccg

SEQ ID NO: 10 - DNA, 32 bp, Artificial Sequence
  Description of Combined DNA/RNA Molecule Synthetic nucleotide sequence
caggcuacgncgtagagcaucantgatccugt

SEQ ID NO: 11 - DNA, 32 bp, Artificial Sequence; identical to SEQ ID NO: 10
  Description of Artificial Sequence Synthetic nucleotide sequence

SEQ ID NO: 12 - RNA, 27 nt, Artificial Sequence
  Description of Artificial Sequence Synthetic nucleotide sequence
cggaaucagugaaugcuuauacauccg

SEQ ID NO: 13 - RNA, 27 nt, Artificial Sequence
  Description of Artificial Sequence Synthetic nucleotide sequence
cggaaucagugaaugcuuauacauccg

(19) United States
(12) Patent Application Publication
(10) Pub. No.: US 2005/0260651 A1
(43) Pub. Date: Nov. 24, 2005

(54) Enhanced biologically active conjugates

(76) Inventors: Pericles Calias, Melrose, MA (US); Gary P. Cook, Westford, MA (US); David T. Shima, Boston, MA (US); Anthony P. Adamis, Boston, MA (US); Yin-Shan Ng, North Billerica, MA (US); Gregory S. Robinson, Wilmington, MA (US); David I. Turner, Norwell, MA (US); Mary A. Ganley, Norwood, MA (US)

Correspondence Address: EYETECH PHARMACEUTICALS, INC., 3 TIMES SQUARE 12TH FLOOR, NEW YORK, NY, 10036, US

(21) Appl. No.: 11/105,279

(22) Filed: Apr. 13, 2005

Related U.S. Application Data
(60) Provisional application No. 60/561,601, filed on Apr. 13, 2004.
(60) Provisional application No. 60/658,819, filed on Mar. 4, 2005.

(51) Int. Cl. C12Q 1/68; C08G 63/48

(52) U.S. Cl. 435/6; 525/54.2

(57) ABSTRACT

The invention provides compositions and methods for making and using sterically enhanced antagonist aptamer conjugates that include a nucleic acid sequence having a specific affinity for a target molecule and a soluble, high molecular weight steric group that augments or facilitates the inhibition of binding to, or interaction with, the target molecule binding partner by the target molecule when bound to the aptamer conjugate. The present invention also provides methods and formulations for ocular delivery of a biologically active molecule by attaching a charged moiety to the biologically active molecule and delivering the biologically active molecule by iontophoresis. Iontophoresis of a biologically active molecule that is conjugated to a high molecular weight neutral moiety, in enhanced by substituting the high molecular weight neutral moiety with a charged molecule of comparable size.

### RELATED APPLICATIONS

This Application claims the benefit of U.S. Provisional Application No. 60/561,601, filed on Apr. 13, 2004 and U.S. Provisional Application No. 60/658,819, filed on Mar. 4, 2005. The entire teachings of the above applications are incorporated herein by reference.

### FIELD OF THE INVENTION

The invention relates to aptamers or nucleic acid ligands. More specifically, the invention relates to methods for enhancing or augmenting one or more antagonist properties of an aptamer that targets a protein binding pair, particularly a protein binding pair that may be targeted in the treatment of a disease or disorder (such as a protein binding pair associated with neovascularization or angiogenesis). The present invention also relates to methods and formulations for ocular delivery of a biologically active molecule by attaching a charged molecule to the biologically active molecule and delivering the biologically active molecule by iontophoresis.

### BACKGROUND OF THE INVENTION

Aptamers, or nucleic acid ligands, are nucleic acid molecules that bind specifically to molecules, particularly proteins, through interactions other than classic Watson-Crick base pairs. Like peptides generated by phage display or monoclonal antibodies (MAbs), aptamers are able to specifically bind to a selected target and, thereby, block their targets' ability to function. Appropriate aptamer sequences for targeting a particular target can be elucidated using an in vitro selection process starting from pools of random sequence oligonucleotides using a process called SELEX (for Systematic Evolution of Ligands by EXponential enrichment). SELEX is a combinatorial chemistry methodology in which vast numbers of oligonucleotides are screened rapidly for specific sequences that have appropriate binding affinities and specificities toward any target. Using this process, novel aptamer nucleic acid ligands that are specific for a particular target may be created. Such aptamers adopt a specific three-dimensional conformation that binds to the particular selected target. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (hydrogen bonding, electrostatic complementarily, hydrophobic contacts, steric exclusion, etc.) that drive affinity and specificity in antibody/antigen complexes. Once the appropriate aptamer sequence for binding to a particular target is elucidated, the therapeutic aptamers may be chemically synthesized directly in large quantities independent of the SELEX process.

For example, antagonistic VEGF aptamer inhibitors have been developed which block the action of VEGF (the Vascular Endothelial Growth Factor). The anti-VEGF aptamers are small stable RNA-like molecules that bind with high affinity to the 165 kDa isoform of human VEGF. Such VEGF aptamers have broad clinical utility due to the role of the VEGF ligand in a wide variety of diseases involving angiogenesis, including psoriasis, ocular disorders, collagen vascular diseases and neoplastic diseases. The SELEX process in general, and VEGF aptamers and formulations in particular, are described in, e.g., U.S. Pat. Nos. 5,270,163, 5,475,096, 5,696,249, 5,670,637, 5,811,533, 5,817,785, 5,849,479, 5,859,228, 5,958,691, 6,011,020, 6,051,698, 6,147,204, 6,168,778, 6,426,335, and 6,696,252, the contents of each of which is specifically incorporated by reference herein.

Complexes of aptamers with high molecular weight non-immunogenic and lipophilic compounds have been described. For example, U.S. Pat. No. 6,011,020 discloses forming aptamer complexes with high molecular weight non-immunogenic and lipophilic compounds in order to improve pharmacokinetic properties such as aptamer stability (i.e., to increase the in vivo circulation half-life of the aptamer). In addition, U.S. Pat. No. 6,051,698 discloses high molecular weight, non-immunogenic complexes of aptamers that have a specific affinity for vascular endothelial growth factor (VEGF). While selection of high affinity aptamers that bind to various biological targets and modifications that enhance the in vivo stability of such aptamers have been described, compositions and methods for enhancing the antagonist properties of such aptamers would be useful in increasing the actual therapeutic potential of aptamer technology.

Drug delivery into the eye is challenging because the anatomy, physiology and biochemistry of the eye includes several defensive barriers that render ocular tissues impervious to foreign substances. Techniques used for administering active agents into the eye include systemic routes, intraocular injections, injections around the eye, intraocular implants, and topical applications. Such invasive intraocular administrations are not favorable because they cause patient discomfort and sometimes fear, while risking permanent tissue damage.

Ocular bioavailability of drugs applied topically in formulations such as eye drops is very poor. The absorption of drugs in the eye is severely limited by some protective mechanisms that ensure the proper functioning of the eye, and by other concomitant factors, for example: drainage of the instilled solutions; lacrhymation, tear evaporation; non-productive absorption/adsorption such as conjunctival absorption, poor corneal permeability, binding by the lachrymal proteins, and metabolism.

Alternative approaches to delivery include in situ activated gel-forming systems, mucoadhesive formulations, ocular penetration enhancers and ophthalmic inserts. In situ activated gel-forming systems are liquid vehicles that undergo a viscosity increase upon instillation in the eye, thus favoring pre-corneal retention. Such a change in viscosity can be triggered by a change in temperature, pH or electrolyte composition. Mucoadhesive formulations are vehicles containing polymers that adhere via non-covalent bonds to conjunctival mucin, thus ensuring contact of the medication with the pre-corneal tissues until mucin turnover causes elimination of the polymer. Ocular penetration enhancers are mainly surface active agents that are applied to the cornea to enhance the permeability of superficial cells by destroying the cell membranes and causing cell lysis in a dose-dependent manner. Ophthalmic inserts are solid devices intended to be placed in the conjunctival sac and to deliver the drug at a comparatively slow rate. One such device is Ocusert®, by Alza Corporation, which is a diffusion unit consisting of a drug reservoir enclosed by two release-controlling membranes made of a copolymer. M. F. Saettone provides a review of continued endeavors devoted to ocular delivery. (“Progress and Problems in Ophthalmic Drug Delivery”, Business Briefing: Pharmatech, Future Drug Delivery, 2002, 167-171). Iontophoresis is drug delivery process that uses a local electrical current to introduce an ionic molecule into biological tissues. Iontophoresis may also be referred to as electrotransport, ionic medication, iontotherapy, and electromotive drug administration (EMDA). Iontophoresis provides an “on-demand” delivery of biologically active molecules across a tissue.

Conjugation of high molecular weight PEG to biologically active molecules may, however, hinder the iontophoretic delivery of the biologically active molecules. It is possible that the molecular weight size constraint and complexity of the PEG may limit the applicability of iontophoretic delivery. Therefore, a convenient, patient friendly method of delivering conjugated biologically active molecules, circumventing the protective barriers of the eye without causing permanent tissue damage and patient discomfort, remains elusive. In view of the problems described above, there is a need for methods and formulations for enhancing iontophoretic delivery of biologically active molecules.

### SUMMARY OF THE INVENTION

The invention is based, in part, upon the finding that addition of a soluble, high molecular weight steric group to an aptamer increases the aptamer's intrinsic antagonist properties. In particular, the invention relates to the finding that PEGylated forms of an anti-VEGF aptamer have expanded VEGF receptor (VEGFR) antagonist activities over forms of the aptamer that are not PEGylated. Furthermore, without restricting the invention to a particular theory or mechanism of action, the principle of expanded antagonist activity resulting from steric enhancement of an aptamer is generally applicable to aptamers which effect disruption of a protein/protein interaction (e.g., those which block the interaction of one protein with a binding partner, such as a ligand and its receptor).

Thus in one aspect, the invention provides a method of increasing an antagonist property of an aptamer directed to a ligand or its receptor by joining the aptamer to a soluble, high molecular weight steric group at any position along the aptamer, wherein the soluble, high molecular weight steric group increases at least one antagonist property of the aptamer.

In broader aspects, the sterically enhanced aptamer targets a protein that interacts with a second protein, and the joining of the aptamer sequence to the soluble, high molecular weight steric group results in the an increase in the ability of the aptamer to disrupt the interaction of the protein with the second protein (i.e., the target protein's binding partner). The sterically enhanced aptamer thereby increases an antagonist property of the aptamer directed to a target protein.

In another aspect, the invention provides a method of increasing the receptor antagonist range of a ligand-binding aptamer, where the ligand binds to multiple receptors and where the ligand-binding aptamer fails to effectively antagonize the ligand-dependent activation of at least one of the multiple receptors. In this aspect, the method of invention provides for joining the aptamer to a soluble, high molecular weight steric group, so that the aptamer, when joined to the soluble, high molecular weight steric group, effectively antagonizes the ligand-dependent activation of the one or more receptors that the aptamer nucleic acid sequence alone did not effectively antagonize.

In a related aspect, the invention provides a method of increasing the ligand antagonist range of a receptor-binding aptamer, where the receptor binds to multiple ligands and where the receptor-binding aptamer fails to effectively antagonize the ligand-dependent activation of at least one of the multiple ligands. In this aspect, the method of invention provides for joining the aptamer to a soluble, high molecular weight steric group, so that the aptamer, when joined to the soluble, high molecular weight steric group, effectively antagonizes the ligand-dependent activation of the one or more ligands that is not otherwise effectively antagonized by the aptamer alone.

In certain embodiments, the soluble, high molecular weight steric group is dextran. In other embodiments, the soluble, high molecular weight steric group is polyethylene glycol. In still other particularly useful embodiments, the soluble high molecular weight steric group may be a polysaccharide, a glycosaminoglycan, a hyaluronan, an alginate, a polyester, a high molecular weight polyoxyalkylene ether (such as Pluronic™), a polyamide, a polyurethane, a polysiloxane, a polyacrylate, a polyol, a polyvinylpyrrolidone, a polyvinyl alcohol, a polyanhydride, a carboxymethyl cellulose (CMC), a cellulose derivative, a Chitosan, a polyaldehyde, or a polyether. In particular embodiments the polyester group may be a co-block polymeric polyesteric group. In other embodiments, the alginate group may be an anionic alginate group that is provided as a salt with a cationic counter-ion, such as sodium or calcium. In further embodiments, the polyaldehyde group may be either synthetically derived or obtained by oxidation of an oligosaccharide. In particularly useful embodiments, the soluble high molecular weight steric group is a polymeric composition having a molecular weight of about 20 to about 100 kDa.

In particular useful embodiments of the above aspects of the invention, the aptamer is directed to VEGF-A. In other particular embodiments, the aptamer is directed to VEGF-B, VEGF-C, VEGF-D, or VEGF-E. In still other embodiments, the aptamer is directed to a VEGF receptor, such as Flk-1/KDR (VEGFR-2), Flt-1 (VEGFR-1), or Flt-4 (VEGFR-3). In further embodiments, the aptamer is directed to a VEGF co-receptor, such as a neuropilin (e.g., neuropilin-1 or neuropilin-2). In still other embodiments the VEGF co-receptor targeted by the aptamer is V 3 integrin or VE-cadherin.

In further embodiments, the aptamer is directed to any known ligand or its receptor. In further useful embodiments of the invention, the aptamer is directed to an adhesion molecule, such as ICAM-1, or its binding LFA-1. Examples of ligands and/or their receptors for targeting with the sterically enhanced aptamer conjugates of the invention include, but are not limited to, TGF, PDGF, IGF, and FGF. Further ligands and/or their receptors for targeting include: cytokines, lymphokines, growth factors, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL18, IFN, TNF0, TNF1, TNF2, G-CSF, Meg-CSF, GM-CSF; thrombopoietin, stem cell factor, and erythropoietin, hepatocyte growth factor/NK1 or factors that modulate angiogenesis, such as angiopoietins Ang-1, Ang-2, Ang-4, Ang-Y, and/or the human angiopoietin-like polypeptide, and/or vascular endothelial growth factor (VEGF). Particular other factors for targeting with the compositions of the invention include angiogenin, BMPs such as bone morphogenic protein-1, etc., bone morphogenic protein receptors such as bone morphogenic protein receptors IA and IB, neurotrophic factors, chemotactic factor, CD proteins such as CD3, CD4, CD8, CD19 and CD20; erythropoietin; osteoinductive factors; immunotoxins; bone morphogenetic proteins (BMPs); interferons, such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and fragments, combinations and/or variants of any of the above-listed polypeptides.

The invention further includes compositions comprising any of the known aptamer nucleic acid sequences that target, for example, a ligand or its receptor, such as those compiled in the aptamer database provided by Ellington et al. (Lee J F, Hesselberth J R, Meyers L A, Ellington A D “Aptamer database” Nucleic Acids Research, Jan. 1, 2004;32(Database issue):D95-100).

In certain useful embodiments of the invention, the high molecular weight steric group may be joined to the aptamer at the 5′ end of the aptamer sequence, or at the 3′ end of the aptamer sequence, or at a position other than the 5′ end or 3′ end of the aptamer sequence. Examples of suitable internal aptamer sequence positions for joining to the high molecular weight steric group (i.e., non 5′- or 3′-end positions) include exocyclic amino groups on one or more bases, 5-positions of one or more pyrimidine nucleotides, 8-positions of one or more purine nucleotides, one or more hydroxyl groups of a phosphate, or one or more hydroxyl group of one or more ribose groups of the aptamer nucleic acid sequence.

In another aspect, the invention provides a method of increasing the receptor antagonist range of a VEGF aptamer. In this aspect, the initial VEGF aptamer is a nucleic acid sequence that binds to VEGF, but that fails to effectively antagonize VEGF-dependent activation of at least one VEGF receptor. By this aspect of the invention, the VEGF aptamer is joined to a soluble, high molecular weight steric group so that the resulting VEGF aptamer conjugate effectively antagonizes VEGF-dependent activation of the at least one VEGF receptor that the VEGF aptamer initially failed to effectively antagonize, so that the receptor antagonist range of the VEGF aptamer is thereby increased.

In a related aspect, the invention provides a method of increasing the ligand antagonist range of a VEGFR aptamer. In this aspect, the initial VEGFR aptamer is a nucleic acid sequence that binds to a VEGFR, but that fails to effectively antagonize ligand-dependent activation by at least one VEGF ligand. By this aspect of the invention, the VEGFR aptamer is joined to a soluble, high molecular weight steric group so that the resulting VEGFR aptamer conjugate effectively antagonizes VEGFR-dependent activation by the at least one VEGF ligand that the VEGFR aptamer initially failed to antagonize, so that the ligand antagonist range of the VEGFR aptamer is thereby increased.

In another aspect, the invention provides a method of identifying an aptamer conjugate that has a stronger antagonist effect on a target than the corresponding non-conjugated aptamer. In this aspect of the invention, the target may be a ligand or a receptor of the ligand. The method generally includes the steps of providing multiple aptamer conjugates that are, independently, joined to a soluble, high molecular weight steric group at the 5′ end, at the 3′ end or, optionally, at one or more non 5′-terminal or 3′-terminal positions of the aptamer. Each of these differently-conjugated aptamers is then contacted, independently, with the ligand and the receptor of the ligand and the amount of ligand/receptor binding or ligand-dependent receptor activation in the presence of each aptamer conjugate is compared to the amount of ligand/receptor binding or ligand-dependent receptor activation in the absence of the aptamer conjugate. The particular aptamer conjugate with the greatest ability to inhibit ligand/receptor binding or ligand-dependent receptor activation is then selected. The method thereby identifies an aptamer conjugate having an enhanced antagonist effect on the ligand/receptor target.

In another aspect, the invention provides a method of inhibiting the activity of a site that is separate from the binding site on the ligand or receptor. In this aspect, the invention provides a method of inhibiting the activity of a site separate from to the binding site of an aptamer. In one embodiment, the invention provides a method of inhibiting the activity of a site on a ligand distal to the binding site of an aptamer on the ligand by conjugating a soluble, high molecular weight steric group to the aptamer. An aptamer may bind to a ligand at a region near or adjacent to the active site of the ligand. Addition of a soluble, high molecular weight steric group to the aptamer extends the reach of the aptamer over the adjacent active site; thereby blocking the activity of the ligand.

In another aspect, the invention provides a method of inhibiting the binding of a ligand or receptor at a site that is separate from the binding site on the ligand or receptor. In this aspect, the invention provides a method of inhibiting the binding of a site separate from to the binding site of an aptamer. In one embodiment, the invention provides a method of inhibiting the binding of a target protein to a site on a ligand distal to the binding site of an aptamer on the ligand by conjugating a soluble, high molecular weight steric group to the aptamer. An aptamer may bind to a ligand at a region near or adjacent to the receptor binding site of the ligand. Addition of a soluble, high molecular weight steric group to the aptamer extends the reach of the aptamer over the adjacent receptor binding site; thereby blocking the ability of the ligand to bind to the receptor.

In another aspect, the invention provides a method of inhibiting the binding of a target protein to a binding partner, where the target protein has an acidic domain, which is characterized by an overall negative charge at physiological pH, as well as a basic domain, which is characterized by an overall positive charge a physiological pH. In this aspect of the invention, the binding partner binds through the acidic domain of the target protein and the binding of the target protein to the binding partner is inhibited by contacting the target protein with a sterically enhanced aptamer conjugate that includes an aptamer nucleic acid sequence which binds to the basic domain of the target protein and a soluble, high molecular weight steric group that sterically hinders binding of the binding partner to the acidic domain of the target protein, so that the binding of the target protein to the binding partner is inhibited.

The invention is also based, in part, upon the discovery that the size and neutral charge of polyethylene glycol (PEG) significantly limits iontophoretic delivery of PEGylated biologically active molecules. Applicants have also discovered that substituting the neutral PEG with a charged molecule enhances iontophoretic delivery. The present invention relates to a method of enhancing iontophoresis of a biologically active molecule by attaching a charged molecule to the biologically active molecule.

Thus, in another aspect, the invention relates to a method of delivering a biologically active molecule to an eye comprising the steps of: a) attaching a charged molecule to the biologically active molecule forming a biologically active molecule charged conjugate and b) delivering the biologically active molecule charged conjugate to the eye using iontophoresis.

In one embodiment, the charged molecule comprises a high charge density polymer such as carboxymethyl cellulose (CMC), carboxymethyl dextran (CMD) or chitosan and the biologically active molecule is a nucleic acid such as an aptamer.

In another aspect, the invention relates to formulations useful for iontophoretic delivery of a biologically active molecule to an eye. The formulations comprise a biologically active molecule conjugated to a charged molecule. In one embodiment, the formulations comprise a nucleic acid such as an aptamer conjugated to a high charge density polymer such as CMC, CMD or chitosan.

The iontophoretic delivery methods and formulations of the present invention have several advantages. Highly charged polymers such as CMC or chitosan, act as both a residence time enhancer and iontophoretic facilitator of biologically active molecules. Therefore, the charged molecules facilitate iontophoretic delivery while preserving the extended circulation times of their PEG counterparts. Charged molecules such as CMC and chitosan are widely accepted biocompatible molecules that are available in various molecular weights and have established conjugation chemistries (See Biocompatible Polymers, Metals and Composites, M. Szycher, Technomic Publishing Co., Lancaster, Pa., 1983, which is hereby incorporated by reference in its entirety). The iontophoretic delivery methods and compositions of the present invention provide a non-invasive ocular therapy while considering patient comfort and avoiding permanent tissue damage.

### BRIEF DESCRIPTION OF THE DRAWINGS

FIG. 1 is a schematic representation of the chemical structure of the PEGylated VEGF antagonist aptamer EYE001 (Macugen®, pegaptanib).

FIG. 2 is a schematic representation of the chemical structure of a 5′-5′ capped VEGF antagonist aptamer EYE002 (i.e., Mac II, SEQ ID NO: 1).

FIG. 3(A) is a schematic representation of the polypeptide sequence of a human intercellular adhesion molecule-1 (ICAM-1) precursor corresponding to GenBank Accession No. AAA52709 (SEQ ID NO: 2). The sequence of the 27 amino acid (a.a.) N-terminal signal peptide is shaded, basic amino acid residues in the mature peptide (a.a. 28-532) are shown in bold and acidic amino acid residues in the mature peptide are shown underlined.

FIG. 3(B) is a schematic representation of the nucleotide sequence of a human ICAM-1 encoding nucleic acid sequence corresponding to GenBank Accession No. J03132 (SEQ ID NO: 3). The initiation and termination codons of the ICAM-1 precursor protein open reading frame are underlined.

FIG. 4 is a graphical representation of the results of a VEGFR-1 (Flt-1) inhibition assay using various 5′-PEGylated VEGF aptamer conjugates.

FIG. 5 is a graphical representation of the results of a VEGFR-1 (Flt-1) inhibition assay using various dextran-VEGF aptamer conjugates.

FIG. 6 is a graphical representation of the results of a VEGFR-1 (Flt-1) inhibition assay using various carboxymethyl cellulose (CMC)-VEGF aptamer conjugates.

FIG. 7 is a graphical representation of the results of a VEGFR-1 (Flt-1) inhibition assay using various PEGylated VEGF aptamer conjugates having PEG moieties of various molecular weights and molecular radii (hydrodynamic volumes).

FIG. 8 is a graphical representation of the results of a VEGFR-1 (Flt-1) inhibition assay using various 3′-PEGylated VEGF aptamer conjugates.

FIG. 9 is a schematic representation of a sterically enhanced aptamer bound to a ligand thereby inhibiting the interaction of a ligand and a receptor.

FIG. 10 is a schematic representation of a sterically enhanced aptamer bound to a receptor thereby inhibiting the interaction of a ligand and a receptor.

FIG. 11 is a schematic representation of the design of a sterically enhanced ICAM aptamer antagonist in which an aptamer that binds to a basic region of ICAM (left) is sterically enhanced to effectively block ICAM binding to the ICAM receptor LFA-1 (right).

FIG. 12 is a schematic representation of the general chemical structure of a dextran conjugated aptamer.

FIG. 13 is a schematic representation of the general chemical structure of a carboxymethyl cellulose conjugated aptamer.

FIG. 14 is a schematic representation of the general synthetic method for conjugating BSA to an aptamer.

FIG. 15 is a schematic representation of the general synthetic method for conjugating a dendron to an aptamer.

FIG. 16 is a schematic representation of the general synthetic method for conjugating a bifunctional linker to an aptamer.

### DETAILED DESCRIPTION OF THE INVENTION

The patent and scientific literature referred to herein establishes knowledge that is available to those of skill in the art. All issued patents, patent applications, published foreign applications, and published references, including GenBank database sequences, which are cited herein, are hereby incorporated by reference to the same extent as if each was specifically and individually indicated to be incorporated by reference in their entirety.

General

The invention provides aptamers having enhanced antagonistic activity and methods for increasing the scope of antagonistic activity of site-specific aptamers that bind target proteins that are involved in protein/protein interactions. The invention addresses an inherent limitation of the SELEX methodology, and aptamer design in general, which is that the high negative charge carried by the phosphodiester backbone of nucleic acid aptamers results in preferential selection of aptamer sequences which bind to positively charged regions of the targeted protein (i.e., regions of the target protein that are rich in the basic amino acids arginine, lysine and histidine), regardless of whether such basic regions are critical to protein function (see, e.g., Paborsky et al. (1993) J. Biol. Chem. 268: 20808-11).

Aptamers have a number of desirable characteristics for use as therapeutics including high specificity and affinity, biological efficacy, and excellent pharmacokinetic properties. In addition, they offer specific competitive advantages over antibodies and other protein biologics. These include, for example, the following:
(1) Speed and Control. Aptamers are produced by an entirely in vitro process. In vitro selection allows the specificity and affinity of the aptamer to be tightly controlled and allows the generation of leads against both toxic and non-immunogenic targets.
(2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the I MHC and the immune response is generally trained not to recognize nucleic acid fragments).
(3) Administration. Whereas all currently approved antibody therapeutics are administered by intravenous infusion (typically over 2-4 hours), aptamers can be administered by subcutaneous injection. This difference is primarily due to the comparatively low solubility and thus large volumes necessary for most therapeutic MAbs. With good solubility (>150 mg/mL) and comparatively low molecular weight (aptamer: 10-50 kDa; antibody: 150 kDa), a weekly dose of aptamer may be delivered by injection in a volume of less than 0.5 mL. Aptamer bioavailability via subcutaneous administration is >80% in monkey studies (Tucker, et al. (1999) J. Chromatogr. B. Biomed. Sci. Appl. 732:203-12).
(4) Scalability and Cost. Aptamers are chemically synthesized and consequently can be readily scaled as needed to meet production demand. Whereas difficulties in scaling production are currently limiting the availability of some biologics (e.g., Ebrel, Remicade) and the capital cost of a large-scale protein production plant is enormous (e.g., $500 MM, Immunex), a single large-scale synthesizer can produce upwards of 100 kg oligonucleotide per year and requires a relatively modest initial investment (e.g., <$10 MM, Avecia). The current cost of goods for aptamer synthesis at the kilogram scale is estimated at $500/g, comparable to that for highly optimized antibodies. Continuing improvements in process development are expected to lower the cost of goods to <$ 100 per gram in five years.
(5) Stability. Aptamers are chemically robust. They are intrinsically adapted to regain activity following exposure to heat, denaturants, etc. and can be stored for extended periods (>1 yr) at room temperature as lyophilized powders. In contrast, antibodies must be stored refrigerated.
   Definitions

All technical and scientific terms used herein, unless otherwise defined below, are intended to have the same meaning as commonly understood by one of ordinary skill in the art; references to techniques employed herein are intended to refer to the techniques as commonly understood in the art, including variations on those techniques or substitutions of equivalent or later-developed techniques which would be apparent to one of skill in the art. In order to more clearly and concisely describe the subject matter which is the invention, the following definitions are provided for certain terms which are used in the specification and appended claims.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “alginate,” refers to a hydrophilic polysaccharide that occurs in brown algae (brown seaweeds, e.g., California giant kelp (Macrocystis pyrifera)) and has an interrupted structure of stretches of alpha1-4-linked alpha-L-glopyranosyluronic acid residues, stretches of beta1-4-linked beta-D-mannopyranosyluronic acid residues, and stretches where both uronic acids occur in alternating sequences.

The term “anion” refers to an atom or molecule which has a negative electrical charge.

As used herein, the term “antagonist”, when applied to an aptamer, refers to the ability to disrupt the interaction of the target protein with a binding partner, wherein the interaction of the target protein with the binding partner is involved in a biological function of the target protein. Accordingly, aptamer antagonists will typically function to inhibit a biological function of the target protein. However, for example, when the target protein interacts with an inhibitor protein binding partner, the aptamer antagonist may disrupt the interaction of the target protein with its inhibitor and thereby effect an activation of the biological function of the target protein that is otherwise inhibited by the inhibitor protein. Therefore, while the aptamer antagonists of the invention will typically inhibit the biological function of the target protein, they may serve to activate the biological function of the target.

As used herein, the term “antagonistic range” refers to increasing or adding an antagonistic action of a biologically active molecule. For example, the “antagonistic range” of an antagonist in increased if the antagonist is able to antagonize one or more additional ligand/receptor interactions supplementary to which the antagonist would have been able to antagonize previously. The antagonistic range may be increased by the addition of a steric conjugate. In one embodiment, the range is determined by the linear and/or hydrodynamic volume of the conjugated moiety.

As used herein, the term “aptamer” means any polynucleotide, or salt thereof, having selective binding affinity for a non-polynucleotide molecule (such as a protein) via non-covalent physical interactions. An aptamer is a polynucleotide that binds to a ligand in a manner analogous to the binding of an antibody to its epitope. The target molecule can be any molecule of interest. An example of a non-polynucleotide molecule is a protein. An aptamer can be used to bind to a ligand-binding domain of a protein, thereby preventing interaction of the naturally occurring ligand with the protein. Aptamers of the invention are optionally modified as described herein by joining the aptamer to a soluble, high molecular weight steric group.

A “biologically active molecule”, “biologically active moiety” or “biologically active agent” can be any substance which can affect any physical or biochemical properties of a biological organism, including but not limited to, viruses, bacteria, fungi, plants, animals, and humans. Biologically active molecules can include any substance intended for diagnosis, cure mitigation, treatment, or prevention of disease in humans or other animals, or to otherwise enhance physical or mental well-being of humans or animals. Examples of biologically active molecules include, but are not limited to, nucleic acids, nucleosides, oligonucleotides, antisense oligonucleotides, RNA, DNA, siRNA, aptamers, antibodies, peptides, proteins, enzymes and porphyrins, small molecule drugs. Other biologically active molecules include, but are not limited to, dyes, lipids, cells, viruses, liposomes, microparticles and micelles. Examples of antibodies include, but are not limited to, VEGF antibodies bevacizumab (Avastin™) and ranizumab (Lucentis™). Examples of aptamers include, but are not limited to, pegaptanib (Macugen®). Examples of porphyrins include, but are not limited to, verteporfin (Visudine®). Examples of steroids include, but are not limited to, anecortave (Retaane®). Classes of biologically active molecules that are suitable for use with the invention include, but are not limited to, antibiotics, fungicides, anti-viral agents, anti-infective agents, anti-inflammatory agents, anti-tumor agents, anti-tubulin agents, cardiovascular agents, anti-anxiety agents, hormones, growth factors, steroidal agents, and the like.

The term “cation” refers to an atom or molecule which has a positive electrical charge.

The term “charged molecule” or “charged moiety” as used herein, refers to any moiety or molecule possessing a formal charge. The charged molecule may be permanently charged by virtue of its inherent structure, or as a result of its covalent bonding to another atom. The charged molecule may also posses a formal charge by virtue of the pH conditions existing of the surrounding environment, such as for example, the environment existing during drug delivery. The charge on the molecule may be either positive (cationic) or negative (anionic). The charge molecule can comprise positive charges or negative charges only. The charged molecule can also comprise a combination of both positive and negative charges. In a particular embodiment, the charged molecule has a net anionic charge. Chemical groups that impart a positive charge to a charged molecule include, but are not limited to, ionizable nitrogen atoms, such as in amino-containing compounds. Chemical groups that impart a negative charge to a charged molecule include, but are not limited to, carboxylate, sulfate, sulfonate, phosphonate or phosphate groups.

A charged molecule or a biologically active molecule charged conjugate are optionally accompanied by one or more “counterions”. Counterions accompanying a charged molecule or a biologically active molecule charged conjugate may be considered to be part of the charged molecule. Counterions for both the charged molecule and the resulting biologically active molecule charged conjugate may result in pharmaceutically acceptable salts. Suitable anionic counterions include, but are not limited to, chloride, bromide, iodide, acetate, methanesulfonate, succinate, and the like. Suitable cationic counterions include, but are not limited to, Na⁺, K⁺, Mg²⁺, Ca²⁺, NH⁴⁺ and organic amine cations. Organic amine cations include, but are not limited to, tetraalkylammonium cations and organic amines, that together with a proton, form a quaternary ammonium cations. Examples of organic amines capable of forming quaternary ammonium cations include, but are not limited to, mono- and di-organic amines, mono- and di-amino acids and mono- and di-amino acid esters, diethanolamine, ethylene diamine, methylamine, ethylamine, diethylamine, triethylamine, glucamine, N-methylglucamine, 2-(4-imidazolyl) ethyl amine), glucosamine, histidine, lysine, arginine, tryptophan, piperazine, piperidine, tromethamine, 6′-methoxy-cinchonan-9-ol, cinchonan-9-ol, pyrazole, pyridine, tetracycline, imidazole, adenosine, verapamil and morpholine.

The term “copolymer” refers to a polymer made from more than one kind of monomer.

The term “covalent bond” refers to the joining of two atoms that occurs when they share a pair of electrons.

The terms “current” and “electrical current,” refers to the conductance of electricity by movement of charged particles. The terms “current” and “electrical current,” is intended to be inclusive and not exclusive. In one embodiment the current is a “direct electrical current,” “direct current,” or “constant current.” In another embodiment the current is an “alternating current,” “alternating electrical current,” “alternating current with direct current offset,” “pulsed alternating current,” or “pulsed direct current.”

The term “dendron” refers to a molecule representing half of a dendrimer structure. A dendron is typically constructed on one half of a dendrimer core or by cleavage of a dendrimer core after construction of the dendrimer. The dendron may be composed of any combination of monomer and surface modifications. Examples of useful monomers include, but are not limited to, polyamidoamine (PAMAM). Examples of useful surface modifications include, but are not limited to, cationic ammonium, N-acyl, and N-carboxymethyl modifications. Alternate surface modifications allow for vastly different properties. For example, the dendron may be polyanionic, polycationic, hydrophobic or hydrophilic. The dendron may be rationally tailored such that the precise number of monomers and surface modification groups are determined by the generation of the dendron (G1, G2, G3, G4, G5, and G6 possessing 4, 8, 16, 32, 64, and 128 groups respectively). The construction of a dendron-biologically active molecule conjugate with 1:1 stoichiometry may be accomplished by reduction of the disulfide in a dendrimer that contains a cystamine core. This reduction results in the formation of a single, orthogonal sulphydryl functionality that may be coupled to any biologically active molecule that has been modified such that it contains a single thiol-reactive group. This may be accomplished by reacting the amine-containing biologically active molecule with a bifunctional linker that contains an amine-reactive group on one terminus and a thiol-reactive group on the other terminus. Examples of disulfide-containing dendritic polymers and dendritic polymer conjugates are found in U.S. Pat. No. 6,020,457; which is hereby incorporated by reference in its entirety.

The term “iontophoresis” refers to the transport of ionizable or charged molecules into or through a barrier, such as a tissue, by an electric current. For example, a drug may be transported to a tissue in a body by iontophoresis by applying the drug to the tissue with an electrode carrying the same charge as the drug while the ground electrode is placed elsewhere on the body to complete the electric circuit. An iontophoretic current is established within a tissue when ions within the tissue are transported as a result of an applied potential. The charged compound is attracted to the electrode of opposite polarity and repulsed by the electrode of similar polarity. As a result, compound transport by this method is directly related to the applied potential and the electrophoretic mobility of the compound. Iontophoresis may also be referred to as iontophoretic delivery, electrotransport, iontohydrokinesis, ionic medication, iontotherapy and electromotive drug administration (EMDA).

The term “elongation” refers to the length a composition may achieve (e.g., a high molecular weight polymeric composition) when it is stretched by pulling. Elongation is typically expressed as the length after stretching divided by the original length.

The term “gel” refers to a crosslinked polymer which has absorbed a large amount of solvent. Crosslinked polymers typically swell appreciably when they absorb solvents.

The term “glycosaminoglycan,” refers to any glycan (i.e., polysaccharide) containing a substantial proportion of aminomonosaccharide residues (e.g., any of various polysaccharides derived from an amino hexose).

The term “hydrodynamic volume” refers to the volume a polymer coil occupies when it is in solution. The “hydrodynamic volume” of a polymer can vary depending on the polymer's molecular weight and how well it interacts with the solvent. For example, every ethylene oxide repeating unit of PEG is known to bind 2-3 water molecules. Hydrodynamic volume may be measured in units of molecular radius.

The term “hydrogen bond,” refers to a very strong attraction between a hydrogen atom which is attached to an electronegative atom, and an electronegative atom which is usually on another molecule. For example, the hydrogen atoms on one water molecule are very strongly attracted to the oxygen atoms on another water molecule.

The term “ion” refers to an atom or molecule which has a positive or a negative electrical charge.

The term “iontophoretic device”, as used herein, refers to a device or apparatus suitable for iontophoretic delivery of a biologically active molecule to a subject. Such iontophoretic devices are well known in the art and are also referred to as “iontophoresis devices” or “electrotransport devices”.

The term “non-peptidic polymer”, as used herein, refers to an oligomer substantially without amino acid residues.

The term “non-nucleic acid polymer”, as used herein, refers to an oligomer substantially without nucleotide residues.

“Ocular delivery” and “ophthalmic delivery” refer to delivery of a compound such as a biologically active molecule to an eye tissue or fluid. “Ocular iontophoresis” refers to iontophoretic delivery to an eye tissue or fluid. Any eye tissue or fluid can be treated using iontophoresis. Eye tissues and fluids include, for example, those in, on or around the eye, such as the vitreous, conjunctiva, cornea, sclera, iris, crystalline lens, ciliary body, choroid, retina and optic nerve.

The term “hydrolytically stable” or “non-hydrolyzable” bond or linkage is used herein to refer to bonds or linkages that are substantially stable in water and substantially do not react with water. For example, a hydrolytically stable linkage does not react under physiological conditions for an extended period of time.

The term “physiologically stable” bond or linkage is used herein to refer to bonds or linkages that are substantially stable against in vivo cleavage or hydrolysis, but may be also stable in the presence of other in vitro agents. A physiologically stable bond or linkage is hydrolytically stable and is stable to physiological processes in a cell, an organ, the skin, a membrane or elsewhere within the body of a patient.

A “physiologically cleavable” bond is one that is cleaved or hydrolyzed in vivo, but may be also cleaved by other in vitro agents. Physiological cleavage may be chemical or enzymatic. Physiological cleavage may occur by the physiological processes in a cell, an organ, the skin, a membrane or elsewhere within the body of a patient.

An “esterase resistant” or “esterase stable” bond or linkage is stable in the presence of an esterase.

The terms “polynucleotide” and “oligonucleotide” are meant to encompass any molecule comprising a sequence of covalently joined nucleosides or modified nucleosides which has selective binding affinity for a naturally-occurring nucleic acid of complementary or substantially complementary sequence under appropriate conditions (e.g., pH, temperature, solvent, ionic strength, electric field strength). Polynucleotides include naturally-occurring nucleic acids as well as nucleic acid analogues with modified nucleosides or internucleoside linkages, and molecules which have been modified with linkers or detectable labels which facilitate conjugation or detection.

As used herein, the term “nucleoside” means any of the naturally occurring ribonucleosides or deoxyribonucleosides: adenosine, cytosine, guanosine, thymosine or uracil.

The term “modified nucleotide” or “modified nucleoside” or “modified base” refer to variations of the standard bases, sugars and/or phosphate backbone chemical structures occurring in ribonucleic (i.e., A, C, G and U) and deoxyribonucleic (i.e., A, C, G and T) acids. For example, Gₘ represents 2′-methoxyguanylic acid, Aₘ represents 2′-methoxyadenylic acid, C_{f} represents 2′-fluorocytidylic acid, U_{f} represents 2′-fluorouridylic acid, Aᵣ, represents riboadenylic acid. The aptamer includes cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4-ethanocytosine, phenoxazine cytidine, phenothiazine cytidine, carbazole cytidine or pyridoindole cytidine. The aptamer further includes guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanine, 7-methylguanine, 8-azaguanine, 7-deazaguanine or 3-deazaguanine. The aptamer further includes adenine or any adenine-related base including 6-methyladenine, N6-isopentenyladenine, N6-methyladenine, 1-methyladenine, 2-methyladenine, 2-methylthio-N6-isopentenyladenine, 8-haloadenine (e.g., 8-fluoroadenine, 8-bromoadenine, 8-chloroadenine, and 8-iodoadenine), 8-aminoadenine, 8-sulfhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-aminoadenine, 8-azaadenine, 7-deazaadenine or 3-deazaadenine. Also included is uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N-2-carboxypropyl)uracil, 5-methylaminomethyluracil, 5-propynyl uracil, 6-azouracil, or 4-thiouracil. Examples of other modified base variants known in the art include, without limitation, those listed at 37 C.F.R. §1.822(p) (1), e.g., 4-acetylcytidine, 5-(carboxyhydroxylmethyl)uridine, 2′-methoxycytidine, 5-carboxymethylaminomethyl-2-thioridine, 5-carboxymethylaminomethyluridine, dihydrouridine, 2′-O-methylpseudouridine, β-D-galactosylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, β-D-mannosylqueosine, 5-methoxycarbonylmethyluridine, 5-methoxyuridine, 2-methylthio-N6-isopentenyladenosine, N-((9-β-D-ribofuranosyl-2-methylthiopurine-6-yl)carbamoyl)threonine, N-((9-β-D-ribofuranosylpurine-6-yl)N-methyl-carbamoyl)threonine, urdine-5-oxyacetic acid methylester, uridine-5-oxyacetic acid (v), wybutoxosine, pseudouridine, queosine, 2-thiocytidine, 5-methyl-2-thiouridine, 2-thiouridine, 4-thiouridine, 5-methyluridine, N-((9-β-D-ribofuranosylpurine-6-yl)carbamoyl)threonine, 2′-O-methyl-5-methyluridine, 2′-O-methyluridine, wybutosine, 3-(3-amino-3-carboxypropyl)uridine. Nucleotides also include any of the modified nucleobases described in U.S. Pat. Nos. 3,687,808, 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096, and 5,681,941. Examples of modified nucleoside and nucleotide sugar backbone variants known in the art include, without limitation, those having, e.g., 2′ ribosyl substituents such as F, SH, SCH₃, OCN, Cl, Br, CN, CF₃, OCF₃, SOCH₃, SO₂, CH₃, ONO₂, NO₂, N₃, NH₂, OCH₂CH₂OCH₃, O(CH₂)₂ON(CH₃)₂, OCH₂OCH₂N(CH₃)₂, O(C₁₋₁₀ alkyl), O(C₂₋₁₀ alkenyl), O(C₂₋₁₀ alkynyl), S(C₁₋₁₀ alkyl), S(C₂₋₁₀ alkenyl), S(C₂₋₁₀ alkynyl), NH(C₁₋₁₀ alkyl), NH(C₂₋₁₀ alkenyl), NH(C₂₋₁₀ alkynyl), and O-alkyl-O-alkyl. Desirable 2′ ribosyl substituents include 2′-methoxy (2′-OCH₃), 2′-aminopropoxy (2′ OCH₂CH₂CH₂NH₂), 2′-allyl (2′-CH₂—CH═CH₂), 2′-O-allyl (2′-O—CH₂—CH═CH₂), 2′-amino (2′-NH₂), and 2′-fluoro (2′-F). The 2′-substituent may be in the arabino (up) position or ribo (down) position.

As used herein, the term “5′-5′ inverted nucleotide cap” means a first nucleotide covalently linked to the 5′ end of an oligonucleotide via a phosphodiester linkage between the 5′ position of the first nucleotide and the 5′ terminus of the oligonucleotide as shown below.

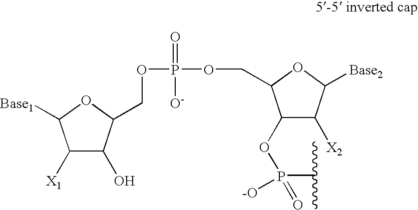

The term “3′-3′ inverted nucleotide cap” is used herein to mean a last nucleotide covalently linked to the 3′ end of an oligonucleotide via a phosphodiester linkage between the 3′ position of the last nucleotide and the 3′ terminus of the oligonucleotide as shown below.

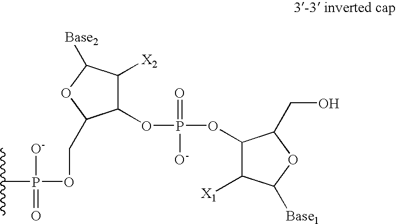

Aptamer compositions, may include, but are not limited to, those having 5′-5′ inverted nucleotide cap structures, those having 3′-3′ inverted nucleotide cap structures, and those having both 5′-5′ and 3′-3′ inverted nucleotide cap structures at the aptamer ends.

“Anti-VEGF aptamers” are meant to encompass polynucleotide aptamers that bind to, and inhibit the activity of, VEGF. Such anti-VEGF aptamers may be RNA aptamers, DNA aptamers or aptamers having a mixed (i.e., both RNA and DNA) composition. Such aptamers can be identified using known methods. For example, Systematic Evolution of Ligands by Exponential enrichment, or SELEX, methods can be used as described in U.S. Pat. Nos. 5,475,096 and 5,270,163, each of which are incorporated herein by reference in its entirety. Anti-VEGF aptamers include the sequences described in U.S. Pat. Nos. 6,168,778, 6,051,698, 5,859,228, and 6,426,335, each of which are incorporated herein by reference in its entirety. The sequences can be modified to include 5′-5′ and/or 3′-3′ inverted caps. (See Adamis, A. P. et al., published application No. WO 2005/014814, which is hereby incorporated by reference in its entirety).

Suitable anti-VEGF aptamer sequences of the invention include the nucleotide sequence GAAGAAUUGG (SEQ ID NO: 4); or the nucleotide sequence UUGGACGC (SEQ ID NO: 5); or the nucleotide sequence GUGAAUGC (SEQ ID NO: 6).

Examples of anti-VEGF aptamers include, but are not limited to:
(i) An anti-VEGF aptamer having the sequence: CGGAAUCAGUGAAUGCUUAUACAUCCG (SEQ ID NO: 7 described in U.S. Pat. No. 6,051,698, incorporated herein by reference in its entirety). Each C, G, A, and U represents, respectively, the naturally-occurring nucleotides cytidine, guanidine, adenine, and uridine, or modified nucleotides corresponding thereto; and preferably

(ii) An anti-VEGF aptamer having the sequence:

(SEQ ID NO: 8)

C_{f}GₘGₘAᵣAᵣU_{f}C_{f}AₘGₘU_{f}GₘAₘAₘU_{f}GₘC_{f}U_{f}U_{f}AₘU_{f}AₘC_{f}AₘU_{f}C_{f}
C_{f}Gₘ

An example of a capped anti-VEGF aptamer has the sequence:

(SEQ ID NO: 9)

X-5′-5′-CGGAAUCAGUGAAUGCUUAUACAUCCG-3′-3′-X

where each C, G, A, and U represents, respectively, the naturally-occurring nucleotides cytidine, guanidine, adenine, and uridine, or modified nucleotides corresponding thereto; X-5′-5′ is an inverted nucleotide capping the 5′ terminus of the aptamer; 3′-3′-X is an inverted nucleotide capping the 3′ terminus of the aptamer; and the remaining nucleotides or modified nucleotides are sequentially linked via 5′-3′ phosphodiester linkages. In some embodiments, each of the nucleotides of the capped anti-VEGF aptamer, individually carries a 2′ ribosyl substitution, such as -OH (which is standard for ribonucleic acids (RNAs)), or —H (which is standard for deoxyribonucleic acids (DNAs)). In other embodiments the 2′ ribosyl position is substituted with an O(C₁₋₁₀ alkyl), an O(C₁₋₁₀ alkenyl), a F, an N₃, or an NH₂ substituent.

In a still more particular non-limiting example, the 5′-5′ capped anti-VEGF aptamer may have the structure:

(SEQ ID NO: 1)

T_{d}-5′-5′-C_{f}GₘGₘAᵣAᵣU_{f}C_{f}AₘGₘU_{f}GₘAₘAₘU_{f}GₘC_{f}U_{f}U_{f}AₘU_{f}
AₘC_{f}AₘU_{f}C_{f}C_{f}G_{m 3′-3′-T}_{d}

wherein “Gm” represents 2′-methoxyguanylic acid, “Aₘ” represents 2′-methoxyadenylic acid, “C_{f}” represents 2′-fluorocytidylic acid, “U_{f}” represents 2′-fluorouridylic acid, “Aᵣ” represents riboadenylic acid, and “Td” represents deoxyribothymidylic acid. (See Adamis, A. P. et al., published application No. WO 2005/014814, which is hereby incorporated by reference in its entirety.)

“Anti-PDGF aptamers” are meant to encompass polynucleotide aptamers that bind to, and inhibit the activity of, PDGF. Such aptamers can be identified using known methods. For example, Systematic Evolution of Ligands by Exponential enrichment, or SELEX, methods can be used as described above.

Anti-PDGF aptamers include the sequences described in U.S. Pat. Nos. 5,668,264, 5,674,685, 5,723,594, 6,229,002, 6,582,918, and 6,699,843 which can be modified, in accordance with the present invention, to include 5′-5′ and/or 3′-3′ inverted caps and/or modifications with a soluble, high molecular weight steric group.

Examples of Anti- PDGF aptamers include, but are not limited to:
(i) ARC-127 (Archemix Corp., Cambridge, Mass.), a PEGylated, anti-PDGF aptamer having the sequence CAGGCUACGN CGTAGAGCAU CANTGATCCU GT (SEQ ID NO: 10 from U.S. Pat. No. 6,582,918, incorporated herein by reference in its entirety) having 2′-fluoro-2′-deoxyuridine at positions 6, 20 and 30, 2′-fluoro-2′-deoxycytidine at positions 8, 21, 28, and 29, 2′-O-Methyl-2′-deoxyguanosine at positions 9, 15, 17, and 31, 2′-O-Methyl-2′-deoxyadenosine at position 22, hexaethylene-glycol phosphoramidite at “N” in positions 10 and 23, and an inverted orientation T (i.e., 3′-3′-linked) at position 32.
   and
(ii) CAGGCUACGN CGTAGAGCAU CANTGATCCU GT (SEQ ID NO: 11 from U.S. Pat. No. 5,723,594, incorporated herein by reference in its entirety) having O-methyl-2-deoxycytidine at C at position 8, 2-O-methyl-2-deoxyguanosine at Gs at positions 9, 17 and 31, 2 -O-methyl-2-deoxyadenine at A at position 22, 2-O-methyl-2-deoxyuridine at position 30, 2-fluoro-2-deoxyuridine at U at positions 6 and 20, 2-fluoro-2-deoxycytidine at C at positions 21, 28 and 29, a pentaethylene glycol phosphoramidite spacer at N at positions 10 and 23, and an inverted orientation T (i.e., 3′-3′-linked) at position 32.

“Anti-ICAM aptamers,” are meant to encompass polynucleotide aptamers that bind to, and inhibit the activity of, ICAM. Such aptamers can be identified using known methods. For example, Systematic Evolution of Ligands by Exponential enrichment, or SELEX, methods can be used as described above.

Unless specifically indicated otherwise, the word “or” is used herein in the inclusive sense of “and/or” and not the exclusive sense of “either/or.”

As used herein, the terms “increase” and “decrease” mean, respectively, a statistically significantly increase (i.e., p<0.1) and a statistically significantly decrease (i.e., p<0.1).

The recitation of a numerical range for a variable, as used herein, is intended to convey that the invention may be practiced with the variable equal to any of the values within that range. Thus, for a variable that is inherently discrete, the variable can be equal to any integer value within the numerical range, including the end-points of the range. Similarly, for a variable that is inherently continuous, the variable can be equal to any real value within the numerical range, including the end-points of the range.

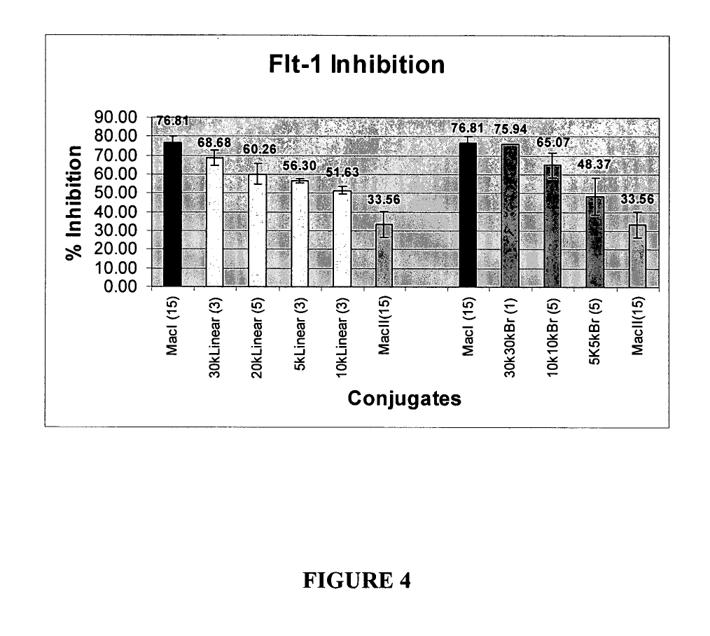

The term “ICAM,” or “intercellular adhesion molecule,” refers to any of several type I membrane glycoproteins of the immunoglobulin superfamily. ICAMs act as ligands for leukocyte adhesion to target cells, in conjunction with LFA-1. LFA-1/ICAM interactions mediate adhesion between many cell types. There are three subclasses of ICAM. ICAM-1 (CD54), has a molecular mass of 90-115 kDa (see FIG. 4(A)) and is expressed on B and T cells, endothelial, epithelial, and dendritic cells as well as fibroblasts, keratinocytes, and chondrocytes. They are inducible in 12-24 hours by cytokines including gamma interferon, interleukin-1β, and tumor necrosis factor-α. Examples of ICAM-1 include ICA1_HUMAN, 532 amino acids (57.76 kDa). ICAM-2 (CD102), has a molecular mass of about 55-65 kDa and is constitutively expressed on endothelial cells, some lymphocytes, monocytes and dendritic cells. Examples of ICAM-2 include ICA2_HUMAN, 275 amino acids (30.62 kDa). ICAM-3 (CD50) has a molecular mass of 116-140 kDa, and is constitutively expressed on monocytes, granulocytes and lymphocytes. Upon physiological stimulation, ICAM-3 becomes rapidly and transiently phosphorylated on serine residues. Examples of ICAM-3 include ICA3_HUMAN, 547 amino acids (59.32 kDa).

The term “oligomer,” as used herein, refers to a polymer whose molecular weight is too low to be considered a polymer. Oligomers typically have molecular weights in the hundreds, but polymers typically have molecular weights in the thousands or higher.

The term “oligonucleotide” refers to an oligomer or polymer of nucleotide or nucleoside monomers consisting of naturally occurring bases, sugars and inter-sugar (backbone) linkages. The term also includes modified or substituted oligomers comprising non-naturally occurring monomers or portions thereof, which function similarly. Incorporation of substituted oligomers is based on factors including enhanced cellular uptake, or increased nuclease resistance and are chosen as is known in the art. The entire oligonucleotide or only portions thereof may contain the substituted oligomers.

The term “polyethylene glycol,” or “PEG” refers to any polymer of general formula H(OCH₂CH₂)ₙOH, wherein n is greater than 3. In one embodiment, n is from about 4 to about 4000. In another embodiment, n is from about 20 to about 2000. In one embodiment, n is about 450. In one embodiment, PEG has a molecular weight of from about 800 Daltons (Da) to about 100,000 Da. In further embodiments, the polyethylene glycol is a 20 kDa PEG, 40 kDa PEG, or 80 kDa PEG. The average relative molecular mass of a polyethylene glycol is sometimes indicated by a suffixed number. For example, a PEG having a molecular weight of 4000 daltons (Da) may be referred to as “polyethylene glycol 4000”). A PEG-conjugated product may be referred to as a PEGylated product.

The term “random coil” refers to the shape of a polymer molecule when its in solution, and it is folded back on itself, rather than being stretched out in a line. Such a random coil forms when the intermolecular forces between the polymer and the solvent are equal to the forces between the solvent molecules themselves and the forces between polymer chain segments.

The term “steric hindrance” refers to the restriction or prevention of the binding or interaction of one molecular entity (e.g., a protein) with another (e.g., an interacting protein). The term “steric hindrance” includes the effect of sterically enhanced aptamers having a soluble, high molecular weight steric group, in restricting or preventing the binding of an aptamer's target protein with the target protein's binding partner (e.g., a ligand with its receptor) due to the sizes and/or spatial disposition of atoms or groups in the steric group.

A “separate site” or “site that is separate from the aptamer binding site” may be proximal or distal to the aptamer binding site. A separate site may be adjacent to, overlapping with, nearby to, or away from the aptamer binding site.

Aptamer Nucleic Acid Compositions

Aptamers nucleic acid sequences are readily made that bind to a wide variety of target molecules. The aptamer nucleic acid sequences of the invention can be comprised entirely of RNA or partially of RNA, or entirely or partially of DNA and/or other nucleotide analogs. Aptamers are typically developed to bind particular ligands by employing known in vivo or in vitro (most typically, in vitro) selection techniques known as SELEX (Systematic Evolution of Ligands by Exponential Enrichment). Methods of making aptamers are described in, for example, Ellington and Szostak (1990) Nature 346:818, Tuerk and Gold (1990) Science 249:505, U.S. Pat. No. 5,582,981; PCT Publication No. WO 00/20040; U.S. Pat. No. 5,270,163; Lorsch and Szostak (1994) Biochem.33:973; Mannironi et al., (1997) Biochem. 36:9726; Blind (1999) Proc. Nat'l. Acad. Sci. USA 96:3606-3610; Huizenga and Szostak (1995) Biochem. 34:656-665; PCT Publication Nos. WO 99/54506, WO 99/27133, and WO 97/42317; and U.S. Pat. No. 5,756,291.

Generally, in their most basic form, in vitro selection techniques for identifying RNA aptamers involve first preparing a large pool of DNA molecules of the desired length that contain at least some region that is randomized or mutagenized. For instance, a common oligonucleotide pool for aptamer selection might contain a region of 20-100 randomized nucleotides flanked on both ends by an about 15-25 nucleotide long region of defined sequence useful for the binding of PCR primers. The oligonucleotide pool is amplified using standard PCR techniques. The DNA pool is then transcribed in vitro. The RNA transcripts are then subjected to affinity chromatography. The transcripts are most typically passed through a column or contacted with magnetic beads or the like on which the target ligand has been immobilized. RNA molecules in the pool which bind to the ligand are retained on the column or bead, while nonbinding sequences are washed away. The RNA molecules which bind the ligand are then reverse transcribed and amplified again by PCR (usually after elution). The selected pool sequences are then put through another round of the same type of selection. Typically, the pool sequences are put through a total of about three to ten iterative rounds of the selection procedure. The cDNA is then amplified, cloned, and sequenced using standard procedures to identify the sequence of the RNA molecules which are capable of acting as aptamers for the target ligand.

For use in the present invention, the aptamer may be selected for ligand binding in the presence of salt concentrations and temperatures which mimic normal physiological conditions. Once an aptamer sequence has been successfully identified, the aptamer may be further optimized by performing additional rounds of selection starting from a pool of oligonucleotides comprising the mutagenized aptamer sequence.

One can generally choose a suitable ligand without reference to whether an aptamer is yet available. In most cases, an aptamer can be obtained which binds the small, organic molecule of choice by someone of ordinary skill in the art. The unique nature of the in vitro selection process allows for the isolation of a suitable aptamer that binds a desired ligand despite a complete dearth of prior knowledge as to what type of structure might bind the desired ligand.

The association constant for the aptamer and associated ligand is, for example, such that the ligand functions to bind to the aptamer and have the desired effect at the concentration of ligand obtained upon administration of the ligand. For in vivo use, for example, the association constant should be such that binding occurs below the concentration of ligand that can be achieved in the serum or other tissue (such as ocular vitreous fluid). For example, the required ligand concentration for in vivo use is also below that which could have undesired effects on the organism.

The aptamer nucleic acid sequences, in addition to including RNA, DNA and mixed compositions, may be modified. For example, certain modified nucleotides can confer improved characteristic on high-affinity nucleic acid ligands containing them, such as improved in vivo stability or improved delivery characteristics. Examples of such modifications include chemical substitutions at the ribose and/or phosphate and/or base positions. SELEX-identified nucleic acid ligands containing modified nucleotides are described in U.S. Pat. No. 5,660,985, entitled “High Affinity Nucleic Acid Ligands Containing Modified Nucleotides,” that describes oligonucleotides containing nucleotide derivatives chemically modified at the 5′ and 2′-positions of pyrimidines. U.S. Pat. No. 5,637,459, supra, describes highly specific nucleic acid ligands containing one or more nucleotides modified with 2′-amino (2′-NH₂), 2′-fluoro (2′-F), and/or 2′-O-methyl (2′-OMe). U.S. application Ser. No. 08/264,029, filed Jun. 22, 1994, entitled “Novel Method of Preparation of Known and Novel 2′ Modified Nucleosides by Intramolecular Nucleophilic Displacement,” describes oligonucleotides containing various 2′-modified pyrimidines.

The aptamer nucleic acid sequences of the invention further may be combined with other selected oligonucleotides and/or non-oligonucleotide functional units as described in U.S. Pat. No. 5,637,459, entitled “Systematic Evolution of Ligands by Exponential Enrichment: Chimeric SELEX,” and U.S. Pat. No. 5,683,867, entitled “Systematic Evolution of Ligands by Exponential Enrichment: Blended SELEX,” respectively.

Antagonist Aptamer Targets

The invention provides aptamers, and more particularly sterically enhanced aptamers conjugated to one or more soluble, high molecular weight steric groups, that function to inhibit the binding of any of various biological targets to one or more binding partners. The aptamer thereby functions as an antagonist of the biological target. In most instances, the disruption of the target/binding partner interaction will function to inhibit one or more biological functions of the target protein. However in certain instances, such as where the binding partner serves to inhibit a biological function of the target, the sterically enhanced aptamer antagonist may activate the biological function of the target protein. Accordingly the “antagonist” aptamer conjugates of the invention are fundamentally “antagonists” of binding between, for example, a target protein (such as a signaling ligand polypeptide) and one or more of its binding partners (such as a cell surface receptor protein).

For example, VEGF aptamer inhibitors have broad clinical utility due to the role of VEGF in a wide variety of diseases involving angiogenesis, including psoriasis, ocular disorders, collagen vascular diseases and neoplastic diseases.

The VEGF ligand occurs in four forms (VEGF-121, VEGF-165, VEGF-189, VEGF-206) as a result of alternative splicing of the VEGF gene (Houck et al. (1991) Mol. Endocrin. 5:1806-1814; Tischer et al. (1991) J. Biol. Chem. 266:11947-11954). The two smaller forms are diffusible whereas the larger two forms remain predominantly localized to the cell membrane as a consequence of their high affinity for heparin. VEGF-165 also binds to heparin and is the most abundant form. VEGF-121, the only form that does not bind to heparin, appears to have a lower affinity for VEGF receptors (Gitay-Goren et al. (1996) J. Biol. Chem. 271:5519-5523) as well as lower mitogenic potency (Keyt et al. (1996) J. Biol. Chem. 271:7788-7795). The biological effects of VEGF are mediated by two tyrosine kinase receptors (Flt-1 and Flk-1/KDR, also known as VEGF-R1 and VEGF-R2 respectively) whose expression is highly restricted to cells of endothelial origin (de Vries et al. (1992) Science 255:989-991; Millauer et al. (1993) Cell 72:835-846; Terman et al. (1991) Oncogene 6:519-524). While the expression of both functional receptors is required for high affinity binding, the chemotactic and mitogenic signaling in endothelial cells appears to occur primarily through the KDR receptor (Park et al. (1994) J. Biol. Chem. 269:25646-25654; Seetharam et al. (1995) Oncogene 10:135-147; Waltenberger et al. (1994) J. Biol. Chem. 26988-26995). The importance of VEGF and VEGF receptors for the development of blood vessels has recently been demonstrated in mice lacking a single allele for the VEGF gene (Carmeliet et al. (1996) Nature 380:435-439; Ferrara et al. (1996) Nature 380:439-442) or both alleles of the Flt-1 (VEGF-R1) (Fong et al. (1995) Nature 376:66-70) or Flk-1/KDR (VEGF-R2) genes (Shalaby et al. (1995) Nature 376:62-66). In each case, distinct abnormalities in vessel formation were observed resulting in embryonic lethality.

VEGF is produced and secreted in varying amounts by virtually all tumor cells (Brown et al. (1997) Regulation of Angiogenesis (Goldberg and Rosen, Eds.) Birkhauser, Basel, pp. 233-269). Direct evidence that VEGF and its receptors contribute to tumor growth was recently obtained by a demonstration that the growth of human tumor xenografts in nude mice could be inhibited by neutralizing antibodies to VEGF (Kim et al. (1993) Nature 362:841-844), by the expression of dominant-negative VEGF receptor flk-1 (Millauer et al. (1996) Cancer Res. 56:1615-1620; Millauer et al. (1994) Nature 367:576-579), by low molecular weight inhibitors of Flk-1 tyrosine kinase activity (Strawn et al. (1966) Cancer Res. 56:3540-3545), or by the expression of antisense sequence to VEGF mRNA (Saleh et al. (1996) Cancer Res. 56:393-401). Importantly, the incidence of tumor metastases was also found to be dramatically reduced by VEGF antagonists (Claffey et al. (1996) Cancer Res. 56:172-181).

Accordingly, aptamer antagonists of VEGF are useful in the treatment of diseases involving neovascularization. For example, VEGF antagonists have been used to treat neovascular age-related macular degeneration (AMD), a progressive condition characterized by the presence of choroidal neovascularization (CNV) that results in more severe vision loss than any other disease in the elderly population (see Csaky et al. (2003) Ophthalmol. 110: 880-1).

One type of VEGF inhibitor is nucleic acid-based VEGF ligand termed an aptamer. Aptamers are chemically synthesized short strands of nucleic acid that adopt specific three-dimensional conformations and are selected for their affinity to a particular target through a process of in vitro selection referred to as systematic evolution of ligands by exponential enrichment (SELEX). SELEX is a combinatorial chemistry methodology in which vast numbers of oligonucleotides are screened rapidly for specific sequences that have appropriate binding affinities and specificities toward any target. Using this process, novel aptamer nucleic acid ligands that are specific for a particular target may be created.

VEGF aptamer inhibitors have been developed which block the action of VEGF. These anti-VEGF aptamers are small stable RNA-like molecules that bind with high affinity to the 165 kDa isoform of human VEGF. Such VEGF aptamers have broad clinical utility due to the role of the VEGF ligand in a wide variety of diseases involving angiogenesis, including psoriasis, ocular disorders, collagen vascular diseases and neoplastic diseases. The SELEX process in general, and VEGF aptamers and formulations in particular, are described in, e.g., U.S. Pat. Nos. 5,270,163, 5,475,096, 5,696,249, 5,670,637, 5,811,533, 5,817,785, 5,849,479, 5,859,228, 5,958,691, 6,011,020, 6,051,698, 6,147,204, 6,168,778, 6,426,335, and 6,696,252, the contents of each of which is specifically incorporated by reference herein.

Many other aptamer sequences have been developed that target various other biological targets. For example, aptamer sequences have been developed that target PDGF (see U.S. Pat. Nos. 5,668,264, 5,674,685, 5,723,594, 6,229,002, 6,582,918, and 6,699,843), basic FGF (see U.S. Pat. Nos. 5,459,015, and 5,639,868), CD40 (see U.S. Pat. Nos. 6,171,795), TGFβ (see U.S. Pat. Nos. 6,124,449, 6,346,611, and 6,713,616), CD4 (see U.S. Pat. No. 5,869,641), chorionic gonadotropin hormone (see U.S. Pat. Nos. 5,837,456, and 5,849,890), HKGF (see U.S. Pat. Nos. 5,731,424, 5,731,144, 5,837,834, and 5,846,713), ICP4 (see U.S. Pat. No. 5,795,721), HIV-reverse transcriptase (see U.S. Pat. No. 5,786,462), HIV-integrase (see U.S. Pat. Nos. 5,587,468, and 5,756,287), HIV-gag (see U.S. Pat. Nos. 5,726,017), HIV-tat (see U.S. Pat. No. 5,637,461), HIV-RT and HIV-rev (see U.S. Pat. Nos. 5,496,938, and 5,503,978), HIV nucleocapsid (see U.S. Pat. Nos. 5,635,615, and 5,654,151), neutophil elastase (see U.S. Pat. Nos. 5,472,841, and 5,734,034), IgE (see U.S. Pat. Nos. 5,629,155, and 5,686,592), tachykinin substance P (see U.S. Pat. Nos. 5,637,682, and 5,648,214), secretory phospholipase A2 (see U.S. Pat. No. 5,622,828), thrombin (see U.S. Pat. No. 5,476,766), intestinal phosphatase (see U.S. Pat. Nos. 6,280,943, 6,387,635, and 6,673,553), tenascin-C (see U.S. Pat. Nos. 6,232,071, and 6,596,491), as well as to cytokines (see U.S. Pat. No. 6,028,186), seven transmembrane G protein-coupled receptors (see U.S. Pat. No. 6,682,886), DNA polymerases (see U.S. Pat. Nos. 5,693,502, 5,763,173, 5,874,557, and 6,020,130,) complement system proteins (see U.S. Pat. Nos. 6,395,888, and 6,566,343), lectins (see U.S. Pat. Nos. 5,780,228, 6,001,988, 6,280,932, and 6,544,959), integrins (see U.S. Pat. No. 6,331,394), and hepatocyte growth factor/scatter factor (HGF/SP) or its receptor (c-met) (see U.S. Pat. No. 6,344,321). These and other aptamer sequences can be incorporated in the invention. Still many more aptamers that target a desired biological target are possible given the adaptability of the SELEX-based methodology.

Other useful aptamer targets include, but are not limited to, NF-κB, RRE, TAR, gp120 of HIV-1, MAP Kinase, Amyloid fibrils, Onostatin M (OSM), E2F, Agiopoietin-2, Coagulation Factor IXa, Ras-induced Raf activation proteins, Nucleocapsids, tubulin, Hepatitis-C virus (HCV), and spiegelmers (mirror image nucleotides).

Particularly useful aptamer targets of the invention include adhesion molecules and their ligands, many of which have large, multidomain extracellular regions that facilitate cell communications and which are particularly amenable to the methods and compositions of the invention. Adhesion molecules include: the selectins (e.g., L-selectin (CD62L, which binds to sulfated GlyCAM-1, CD34, and MAdCAM-1)), E-selectin (CD62E) and P-selectin (CD62P)); the integrins (e.g., LFA-1 (CD11a), which bind to the ICAMs ICAM-1, ICAM-2 and ICAM-3, and CD11b which binds to ICAM-1, Factor X, iC3b and fibrinogen); the immunoglobulin (Ig) superfamily of proteins including the neural specific IgCAMS such as MAG (myelin-associated glycoprotein), MOG (myelin-oligodendrocyte glycoprotein), and NCAM-1 (CD56) and the systemic IgCAMs such as ICAM-1 (CD54) (which binds to LFA-1, see above), ICAM-2 (CD102), ICAM-3 (CD50), and CD44 (which binds to hyaluronin, anykyrin, fibronectin, MIP1β and osteopontin); as well as the cadherins (such as Cadherins E (1), N (2), BR (12), P (3), R (4), etc. and the Desmocollins, such as Desmocollin 1).

Aptamers may be developed for use in diagnostics (e.g., recognizing human red blood cell ghosts, distinguishing differentiated cells from parental cells in carcinoma cell diagnostics) Aptamers may also be developed for use as biosensors. For example, aptamers may specifically target molecules such as proteins, metabolites, amino acids, and nucleotides (e.g., cholera toxin and staphylococcal enterotoxin).

Steric Groups

The invention provides high molecular weight steric groups that are soluble and that may be conjugated to target-specific aptamer nucleic acid sequence. Conjugation of the steric group may be through the 5′ end of the aptamer nucleic acid, the 3′ end of the aptamer nucleic acid, or any position along the aptamer nucleic acid sequence between the 5′ and 3′ ends. For example, the high molecular weight steric group may be conjugated to the aptamer at an exocyclic amino group on a base, a 5-position of a pyrimidine nucleotide, a 8-position of a purine nucleotide, a hydroxyl group of a phosphate, or a hydroxyl group of a ribose group of the aptamer nucleic acid sequence. Means for chemically linking high molecular weight steric groups to aptamer nucleic acid sequences at these various positions are known in the art and/or exemplified below.

Suitable high molecular weight steric groups generally include any soluble high molecular weight compound that has a sufficient hydrodynamic volume to sterically interfere with the interaction between the aptamer-bound target and its binding partner. Examples include, but are not limited to, polymers, gel-forming compounds and the like. Suitable high molecular weight steric groups can include interpenetrating polymer networks and intrapenetrating polymer networks.

The optimal characteristics of a particular soluble high molecular weight steric group may be determined using the procedures taught herein and the methods and compositions taught herein. Methods for determining optimal steric polymers include the inhibition assays described herein as Examples 8 through 12.

Alternatively, Dynamic Light Scattering can be used to measure the hydrodynamic radius of soluble high molecular weight steric groups. Correlating hydrodynamic radius and efficacy may provide an indirect efficacy measurement.

Examples of particularly useful steric groups of the invention include, but are not limited to, polysaccharides, such as glycosaminoglycans, hyaluronans, and alginates, polyesters, high molecular weight polyoxyalkylene ether (such as Pluronic™), polyamides, polyurethanes, polysiloxanes, polyacrylates, polyols, polyvinylpyrrolidones, polyvinyl alcohols, polyanhydrides, carboxymethyl celluloses, other cellulose derivatives, Chitosan, polyadlehydes or polyethers.

Useful steric groups will be soluble in water or physiological solutions. In one embodiment the steric groups have a water solubility of at least 1 mg/mL. In another embodiment the steric groups have a water solubility of at least 10 mg/mL. In another embodiment the steric groups have a water solubility of at least 100 mg/mL.

Useful steric groups will have a molecular weight ranging from about 800 Da to about 3,000,000 Da, and/or a hydrodynamic volume of sufficient size to provide steric hindrance (e.g, to block binding of the antagonist aptamer target with a target binding partner, such as a ligand with its receptor. In one embodiment the steric groups have a molecular weight of from about 20 kilodaltons (kDa) to about 1000 kDa. In another embodiment the steric groups have a molecular weight from about 5 kDa to about 100 kDa. In one particular embodiment, the steric groups have a molecular weight of about 20 kDa. In another particular embodiment, the steric groups have a molecular weight of about 40 kDa. In another particular embodiment, the steric groups have a molecular weight of about 80 kDa.

In one embodiment the steric groups have a hydrodynamic volume ranging from about 0.5 nanometers (nm) to about 1000 nm. In another embodiment the steric groups have a hydrodynamic volume from about 1 nm to about 10 nm. In one particular embodiment, the steric groups have a hydrodynamic volume of about 2 nm. In another particular embodiment, the steric groups have a hydrodynamic volume of about 4 nm. In another particular embodiment, the steric groups have a hydrodynamic volume of about 8 nm.

In one embodiment, the soluble, high molecular weight steric group is a polyether polyol. In a preferred embodiment, the soluble, high molecular weight steric group is a polyethylene glycol (PEG). PEG may have a free hydroxyl group or may be alkylated. In a preferred embodiment, the terminal end of the PEG not bound to the aptamer has a methoxy group (mPEG).

In another embodiment the soluble, high molecular weight steric group is a polysaccharide. In one embodiment, the soluble, high molecular weight steric group is dextran. Dextran may be linear or branched In one embodiment, The dextran is a Carboxymethyl Dextran (CMDex).

In another embodiment the soluble, high molecular weight steric group is a cellulose derivative. In another embodiment the soluble, high molecular weight steric group is a carboxymethyl cellulose (CMC). CMC, an analog of dextran, and its reducing end is available for coupling to an amine group of a biologically active compound by the Schiff-Base chemistry in conjugation. In another embodiment the soluble, high molecular weight steric group is a polyglucosamine. In another embodiment the soluble, high molecular weight steric group is a Chitosan.

Polysaccharides may be attached to an amine at a terminus of the aptamer by reductive amination. Polysaccharides containing a reducing terminus such as an aldehyde or hemiacetal functionality may be conjugated to a primary amine-containing aptamer by reductive amination to afford a secondary amine linkage. Alternately, an aptamer may be modified such that a covalent linkage exists between the aptamer and a hydrazine or hydrazide functionality. The formation of an imine with either of these amine equivalents provides a conjugate that is stabilized to hydrolysis relative to a conventional imine. The hydrazine or hydrazide couplings are useful when the reductive amination is limited by the length of the linker. For example, a hydrazine or hydrazide coupling is especially useful when a linker is needed to separate a bulky moiety and a high electron density macromolecule moiety, while allowing the reactive group of each moiety to come together. The linker between an oligonucleotide amine and the hydrazine or hydrazide may afford an extra measure of steric freedom. The imine that results from a hydrazine or hydrazide may be used without further reduction or reduced to afford an amine-like linkage.

In another embodiment the soluble, high molecular weight steric group is a polyaldehyde. In further embodiments, the polyaldehyde group may be either synthetically derived or obtained by oxidation of an oligosaccharide.

In another embodiment the soluble, high molecular weight steric group is an alginate. In a preferred embodiment, the alginate group is an anionic alginate group that is provided as a salt with a cationic counter-ion, such as sodium or calcium.

In another embodiment the soluble, high molecular weight steric group is a polyester. In particular embodiments the polyester group may be a co-block polymeric polyesteric group.

In another embodiment the soluble, high molecular weight steric group is a polylactic acid (PLA) or a polylactide-co-glycolide (PLGA). Suitable PLGA groups and method s for conjugating PLGA groups are found in J. H. Jeong et al., Bioconjugate Chemistry 2001, 12, 917-923; J. E. Oh et al., Journal of Controlled Release 1999, 57, 269-280 and J. E. Oh et al., U.S. Pat. No. 6,589,548; the contents of each are hereby incorporated by reference in their entirety.

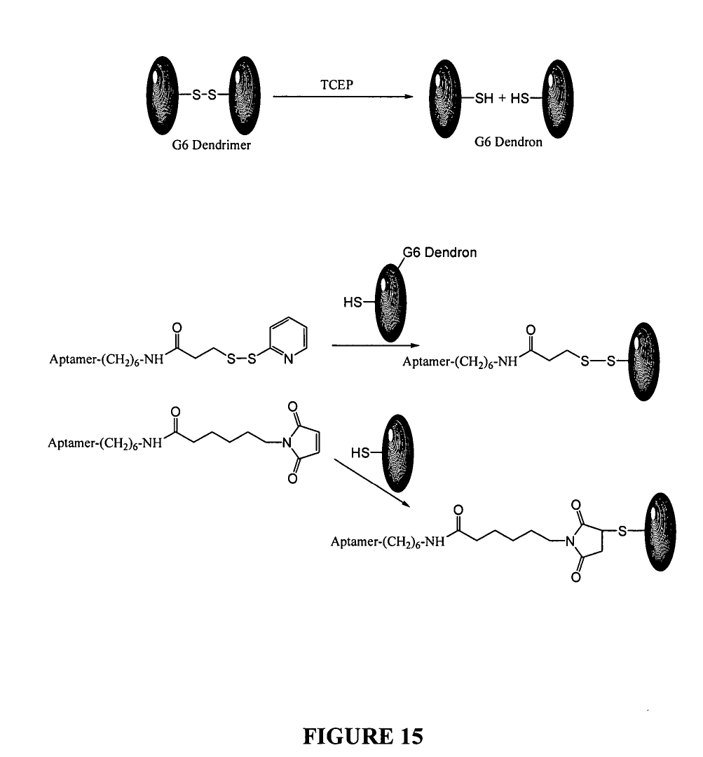

In another embodiment, the high molecular weight steric group is a dendron. The dendron may be composed of any combination of monomer and surface modifications. Examples of useful monomers include, but are not limited to, polyamidoamine (PAMAM). Examples of useful surface modification groups include, but are not limited to, cationic ammonium, N-acyl, and N-carboxymethyl group. The dendron may be polyanionic, polycationic, hydrophobic or hydrophilic. In one particular embodiment, the dendron has about 1 to about 256 surface modification groups. In another particular embodiment, the dendron has about 4, 8, 16, 32, 64 or 128 surface modification groups. Examples of dendron and dendrimer conjugation techniques are found in U.S. Pat. No. 5,714,166; which is hereby incorporated by reference in its entirety. A general synthetic scheme for conjugating a dendron to an aptamer is shown in FIG. 15.

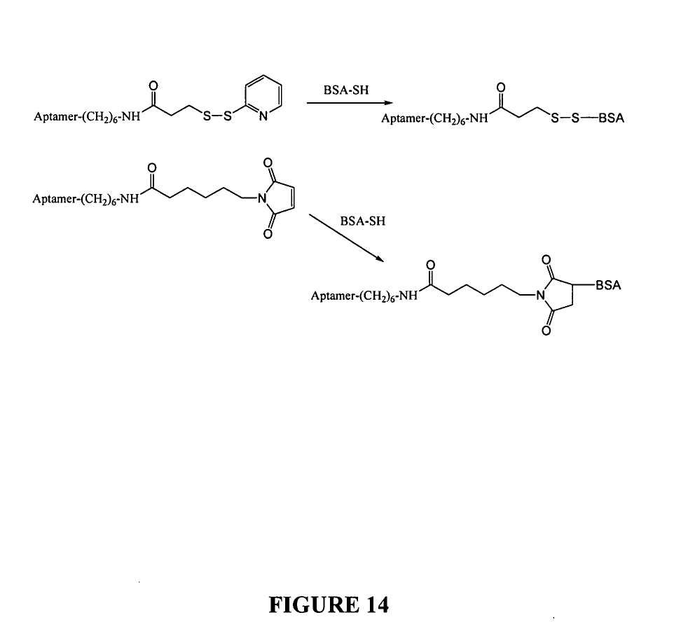

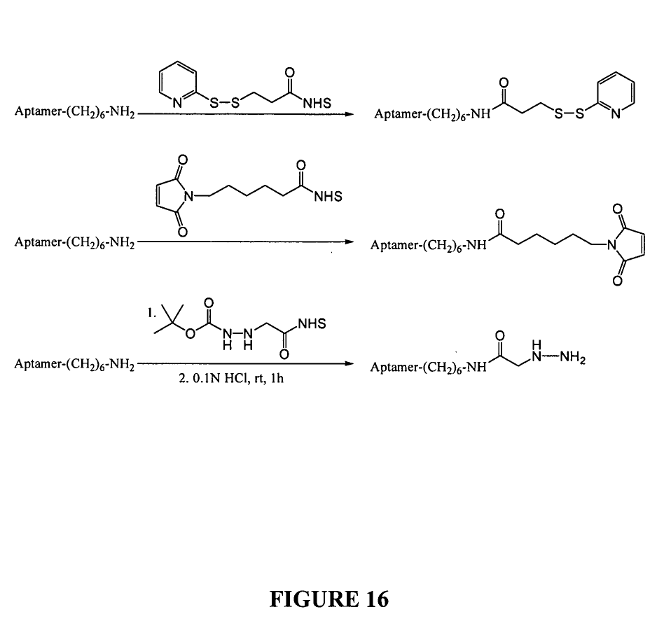

In another embodiment, the soluble, high molecular weight steric group is bovine serum albumin (BSA). The presence of free thiol on BSA permits the conjugation of amine-containing aptamer to BSA by employing a bifunctional linker that contains a thiol-reactive group on one terminus and an amine-reactive group on the other terminus. A general synthetic scheme for conjugating BSA to an aptamer is shown in FIG. 14. A general synthetic scheme for conjugating a bifunctional linker to an aptamer is shown in FIG. 16.

In other particularly useful embodiments the soluble high molecular weight steric group may be a glycosaminoglycan, a hyaluronan, a hyaluronic acid (HA), an alginate a high molecular weight polyoxyalkylene ether (such as Pluronic™), a polyamide, a polyurethane, a polysiloxane, a polyacrylate, a polyvinylpyrrolidone, a polyvinyl alcohol, a polyanhydride, a polyether or a polycaprolactone.

Charged Molecules

The invention provides high charged molecules that may be conjugated to a biologically active molecule such as a target-specific aptamer nucleic acid sequence. The charged molecules can be any suitable charges molecule known in the art. Preferably the charged molecules are anionic or cationic charged polymer or polyelectrolyte. Means for chemically linking the charged molecules to the biologically active molecules are known in the art and/or exemplified below.

Examples of anionic polymers include, but are not limited to, carboxymethyl cellulose (CMC), polyacrylamide, cellulose acetate phthalate (CAP), carrageenan, cellulose sulfate, dextran/dextrin sulfate, poly(naphthalene sulfonate), poly(styrene-4-sulfonate) and poly(4-styrenesulfonic acid-co-maleic acid).

Examples of cationic polymers include, but are not limited to, chitosan, polyglucosamine, polylysine, polyglutamate, polyvinylamine, polymers comprising amines such as 2-(diethylamino)ethanol (DEAE), spermine and putrescine, and other polyamines.

The term “polyelectrolyte” is used to describe any molecule, ion or particle, organic or inorganic, that is charged (negatively charged, positively charged, or zwitterionic), or that is capable of being rendered charged. Polyelectrolytes have at least one, and preferably two or more charged groups. The term “polyelectrolyte” also includes a mixture of different polyelectrolytes or similar polyelectrolytes with different molecular weight distributions. The “polyelectrolyte” may be a single molecule or an aggregate of molecules. If the polyelectrolyte is particulate, i.e., comprised of a plurality of molecular aggregates, the particles can be porous or nonporous, and may be, for example, macromolecular structures such as micelles (cationic or anionic) or liposomes (cationic or anionic). The polyelectrolyte can be selected from the group consisting of cationic polyelectrolytes, anionic polyelectrolytes, amphoteric polyelectrolytes, and mixtures thereof.

Polyelectrolyte can typically comprise a polymer backbone comprising one or more ionic groups selected from the group consisting of quaternary ammonium, sulfonium, phosphonium, carboxylates, sulfonates and phosphates.

Examples of backbone structures suitable for such polyelectrolyte compounds include, but are not limited to, acrylamides, addition polymers (e.g., polystyrenes), oligosaccharides and polysaccharides (e.g., agaroses, dextrans, celluloses), polyamines and polycarboxylic acid salts, polyethylenes, polyimines, polystyrenes, and mixtures thereof. a

Cationic polyelectrolytes typically contain one or more ionic groups such as quaternary ammonium; primary, secondary, or tertiary amines charged at the reservoir solution pH; heterocyclic compounds charged at reservoir solution pH; sulfonium; or phosphonium groups.

Anionic polyelectrolytes typically contain one or more ionic groups such as carboxylate, sulfonate and phosphate groups.

In addition, polyelectrolytes having characteristics of more than one of these categories may also be used in the methods of the invention. For example, partial hydrolysis of a compound such as polyacrylamide produces an amphoteric polyelectrolyte that has both amide (nonionic) and carboxylic acid (anionic) groups.

Examples of cationic polyelectrolytes include, but are not limited to, addition polymers such as polyvinyl alcohol and other polyvinyl compounds such as poly(vinyl 4-alkylpyridinium), poly(vinylbenzyltrimethy-1 ammonium, and polyvinylimine; aminated styrenes; cholestyramine; polyimines such as polyethylenimine; aminated polysaccharides, particularly cross-linked polysaccharides such as dextrans (e.g., dextran carbonates and DEAE dextran); and mixtures thereof.

Examples of anionic polyelectrolytes include, but are not limited to, acrylamides such as acrylamideo methyl propane sulfonates (poly-AMPS), poly(N-tris(hydroxymethyl)methyl methacrylamide and other anionic copolymers of acrylamide; alginate and alginic acid; addition polymers such as homopolymers and copolymers of derivatives of acrylate and methacrylate (e.g., hydroxyl ethyl methacrylates(poly-HEMA), poly(2-DEAE methacrylate)phosphate, and poly(ethyl acrylate-co-maleic anhydride-co-vinyl acetate) sodium; including salts thereof such as sodium polyacrylates); and polystyrenes (e.g., polystyrene sulfonate, sodium polystyrene sulfonate, sodium polystyrene sodium sulfonate (“NaPSS”), and poly(maleic anhydride-co-styrene)2-butoxyethyl ester, ammonium salt); as well as esters and amides thereof having free hydroxyl functionalities; hyaluronate; oligosaccaharides such as the anionically charged cyclodextrans (e.g., sulfobutyl ether .beta.-cyclodextrans); pectic acid; polyacrylic acids (e.g., poly(acrylic acid-do-ethylene) sodium); polysaccharides, particularly cross-linked polysaccharides such as dextrans (e.g., dextran sulfonates and heparin); polystyrenesulfonic acids; polyvinylphosphonic acids; and mixtures thereof.

Other material suitable for use as polyelectrolytes include, but are not limited to, heparin and heparin derivatives; liposomes, both anionic and cationic; micelles, both anionic and cationic; polyamines such as polyvinylpyridine; polyethylenes including chlorosulfonated polyethylene, poly(4-t-butylphenol-co-ethylene oxide-co-formaldehyde) phosphate, polyethyleneaminosteramide ethyl sulfate, poly(ethylene-co-isobutyl acrylate-co-methacrylate) potassium, poly(ethylene-co-isobutyl acrylate-co-methacrylate) sodium, poly(ethylene-co-isobutyl acrylate-co-methacrylate) sodium zinc, poly(ethylene-co-isobutyl acrylate-co-methacrylate) zinc; poly(ethylene-co-methacrylic acid-co-vinyl acetate) potassium; polyethyleneimine, and poly(ethylene oxide-co-formaldehyde-co-4-nonylphenol) phosphate; polysaccharides, including cross-linked polysaccharides such as agaroses, celluloses (e.g., benzoylated naphthoylated diethylaminoethyl (DEAE) cellulose, benzyl DEAE cellulose, triethylaminoethyl (TEAE) cellulose, carboxymethylcellulose, cellulose phosphate, DEAE cellulose, epichlorohydrin triethanolamine cellulose, oxycellulose, sulfoxyethyl cellulose and QAE cellulose), starch, and the like; and mixtures thereof.

A person of ordinary skill in the art would understand the meaning of the term “high charge density polymer”. A “high charge density polymer”, as used herein, refers to a polymer typically recognized in the art to have a substantially high charge density. In one embodiment, the high charge density polymer may have a charge density ranging from about 1 to about 20 milliequivalents per gram (meq/g). In another embodiment, the high charge density polymer has a charge density of at least 5 meq/g. In another embodiment, the high charge density polymer has a charge density of at least 10 meq/g.

The high molecular weight steric group may be joined to the aptamer at any position on the aptamer. In certain useful embodiments of the invention, the high molecular weight steric group may be joined to the aptamer at the 5′-end of the aptamer sequence, or at the 3′-end of the aptamer sequence, or at a position other than the 5′-end or 3-′ end of the aptamer sequence. Examples of suitable internal aptamer sequence positions for joining to the high molecular weight steric group (i.e., non 5′- or 3′-end positions) include exocyclic amino groups on one or more bases, 5-positions of one or more pyrimidine nucleotides, 8-positions of one or more purine nucleotides, one or more hydroxyl groups of a phosphate, or one or more hydroxyl group of one or more ribose groups of the aptamer nucleic acid sequence.

The invention provides a method of identifying an aptamer conjugate that has a stronger antagonist effect on a target than the corresponding non-conjugated aptamer. The method generally includes the following steps:
a) providing multiple aptamer conjugates that are, independently, joined to a soluble, high molecular weight steric group;
b) contacting each of these differently-conjugated aptamers, independently, with the ligand and the receptor of the ligand;
c) comparing the amount of ligand/receptor binding or ligand-dependent receptor activation in the presence of each aptamer conjugate to the amount of ligand/receptor binding or ligand-dependent receptor activation in the absence of the aptamer conjugate.

The particular aptamer conjugate with the greatest ability to inhibit ligand/receptor binding or ligand-dependent receptor activation is then selected. The method thereby identifies an aptamer conjugate having an enhanced antagonist effect on the ligand/receptor target.

In one embodiment, the method of identifying an aptamer conjugate having an enhanced antagonist effect on a target, wherein the target is a ligand or a receptor of the ligand, comprises the steps of, providing multiple aptamer conjugates that are, independently, joined to a soluble, high molecular weight steric group at the 5′ end, the 3′ end and, at one or more non 5′-terminal or 3′-terminal positions of the aptamer, wherein the soluble, high molecular weight steric group has a molecular weight of about 20 to about 100 kDa and is selected from the group consisting of a polysaccharide, a glycosaminoglycan, a hyaluronan, an alginate, a polyester, a high molecular weight polyoxyalkylene ether, a polyamide, a polyurethane, a polysiloxane, a polyacrylate, a polyol, a polyvinylpyrrolidone, a polyvinyl alcohol, a polyanhydride, a carboxymethyl cellulose, a cellulose derivative, a Chitosan, a polyaldehyde, and a polyether; contacting, independently, each of said aptamer conjugates with the ligand and the receptor of the ligand; detecting the amount of ligand/receptor binding or ligand-dependent receptor activation; and selecting the aptamer conjugate with the greatest ability to inhibit ligand/receptor binding or ligand-dependent receptor activation, wherein the aptamer conjugate has a stronger antagonist effect on a ligand/receptor target than the corresponding non-conjugated aptamer.

Without restricting the invention to a particular theory or mechanism of action, the principle of expanded antagonist activity resulting from steric enhancement of an aptamer is generally applicable to aptamers which effect disruption of a protein/protein interaction (e.g., those which block the interaction of one protein with a binding partner, such as a ligand and its receptor).

In a first mechanism of action, an addition of a soluble, high molecular weight steric group to an aptamer can extend the reach of the aptamer over the separate receptor binding site; thereby blocking the ability of the ligand to bind to the receptor.

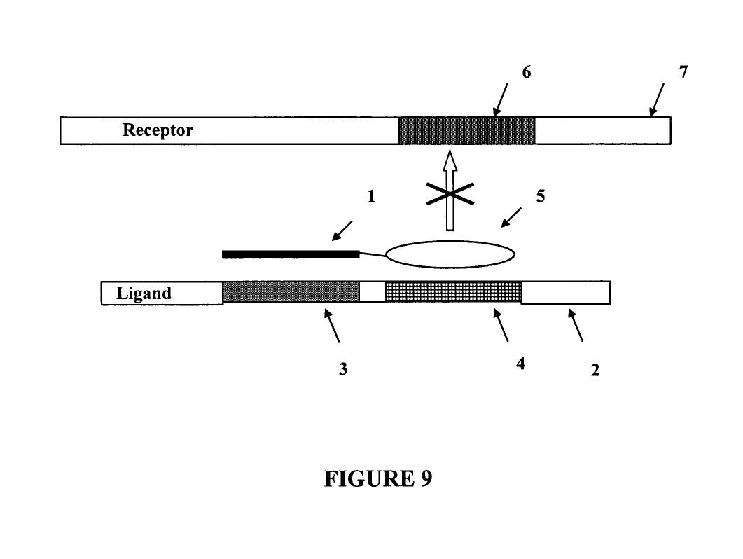

An aptamer may bind to a ligand at a region near, adjacent, proximal or distal to the receptor binding site of the ligand. Addition of a soluble, high molecular weight steric group to the aptamer extends the reach of the aptamer over the adjacent receptor binding site; thereby blocking the ability of the ligand to bind to the receptor. An example of such steric enhancement of an aptamer is shown in FIG. 9. FIG. 9 shows an aptamer (1) that is conjugated to a soluble, high molecular weight steric group (5) binding to a ligand (2) at a site (3) adjacent to the receptor binding site (4) wherein the soluble, high molecular weight steric group (5) extends over the receptor binding site (4). The high molecular weight steric group (5) hinders the ability of receptor binding site (4) of ligand (2) to bind to the ligand binding site (6) of receptor (7).

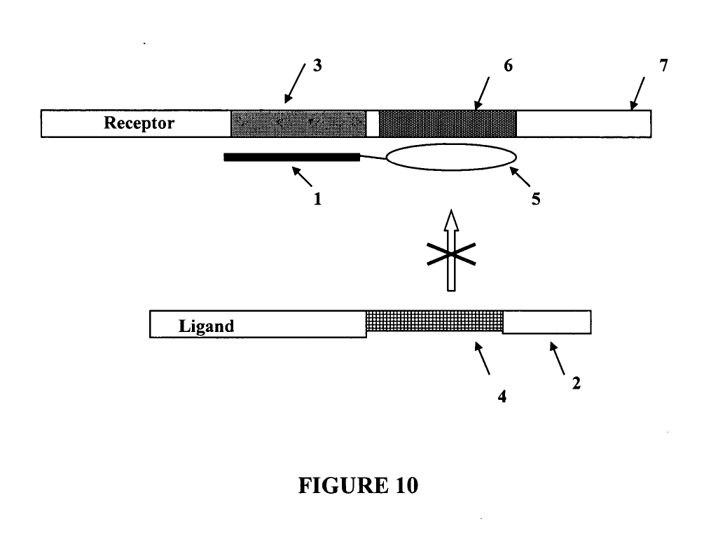

In an analogous manner, an aptamer may bind to a ligand binding receptor at a region near, adjacent, proximal or distal to the ligand binding site of the ligand binding receptor. Addition of a soluble, high molecular weight steric group to the aptamer extends the reach of the aptamer over the adjacent ligand binding site; thereby blocking the ability of the receptor to bind to a ligand. An example of such steric enhancement of an aptamer is shown in FIG. 10. FIG. 10 shows an aptamer (1) that is conjugated to a soluble, high molecular weight steric group (5) binding to receptor (7) at a site (3) adjacent to the ligand binding site (6) wherein the soluble, high molecular weight steric group (5) extends over the ligand binding site (6). The high molecular weight steric group (5) hinders the ability of the receptor binding site (4) of ligand (2) to bind to ligand binding site (6) of receptor (7).

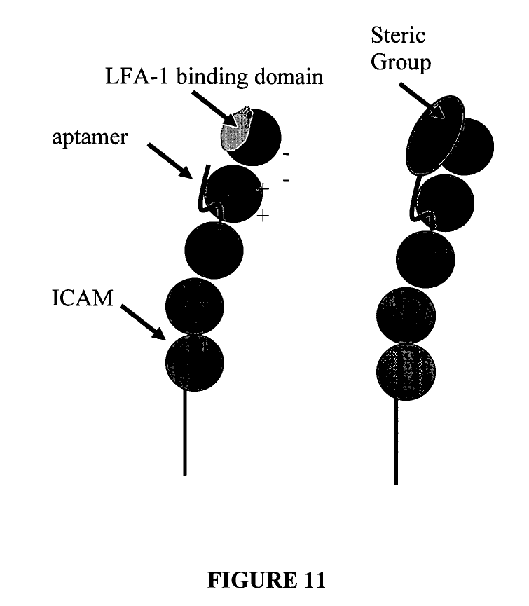

In one aspect of the invention, the sterically enhanced aptamer inhibits the binding of a target protein to a binding partner, where the target protein has an acidic domain that is characterized by an overall negative charge at physiological pH, as well as a basic domain that is characterized by an overall positive charge a physiological pH. In this aspect of the invention, the binding partner binds through the acidic domain of the target protein and the binding of the target protein to the binding partner is inhibited by contacting the target protein with a sterically enhanced aptamer conjugate that includes an aptamer nucleic acid sequence which binds to the basic domain of the target protein and a soluble, high molecular weight steric group that sterically hinders binding of the binding partner to the acidic domain of the target protein, so that the binding of the target protein to the binding partner is inhibited. FIG. 11 is a schematic representation of the design of a sterically enhanced ligand aptamer antagonist in which an aptamer that binds to a basic region of ligand (left) is sterically enhanced to effectively block ligand binding to the ligand receptor (right).

In a second mechanism of action, an addition of a soluble, high molecular weight steric group to the aptamer can elicit an allosteric effect on the ligand. The soluble, high molecular weight steric group may alter the conformation of the ligand, thereby altering the binding activity of the ligand to its receptor. In the case of ligands that have multiple binding sites, allosteric effects can generate cooperative behavior.

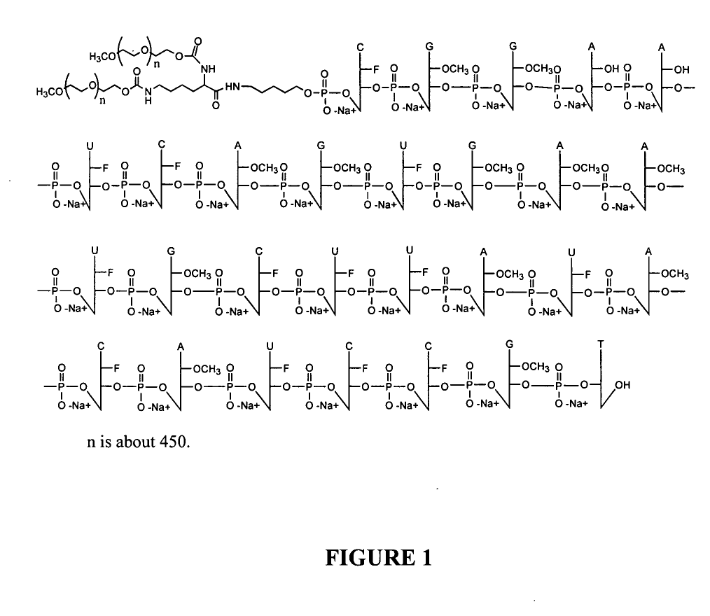

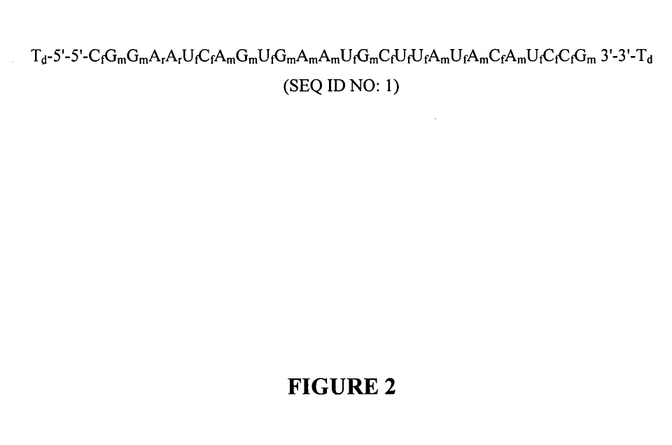

The activity of VEGF aptamers conjugated to soluble, high molecular weight steric groups was determined by a VEGFR-1 (Flt-1) inhibition assay. The results of the assays are shown in FIGS. 4 through 8. The results indicate that sterically enhanced VEGF aptamer conjugates such as Pegaptanib (EYE-001, MacI, the structure of which is shown in FIG. 1) are much more effective in inhibiting VEGF binding than are non-enhanced VEGF aptamers such as EYE-002 (MacII; the structure of which is shown in FIG. 2).

An example of the chemical structure of a 5′-PEGylated aptamer is shown in FIG. 1. A graphical representation of the results of the assay using various 5′-PEGylated VEGF aptamer conjugates are shown in FIG. 4. The effectiveness of the sterically enhanced VEGF aptamer conjugates correlated with the molecular weight of the soluble, high molecular weight steric group that was added. The assays shown in FIG. 4 compared branched PEGs of various molecular weights. For example a conjugate having two 20 kDa PEG units (20K/20K Branched) was compared to a conjugate having two 5 kDa PEG units (5K/5K Branched). The assays shown in FIG. 4 also compared linear PEGs of various molecular weights. For example a conjugate having a 30 kDa PEG (30K Linear) was compared to a conjugate having a 10 kDa PEG (10K Linear). Significantly, non-conjugated PEG alone (control) did not inhibit binding of VEGF to Flt-1 indicating that these soluble, high molecular weight steric groups do not directly affect VEGF/Flt-1 binding, but act through the VEGF aptamer to which they are conjugated.

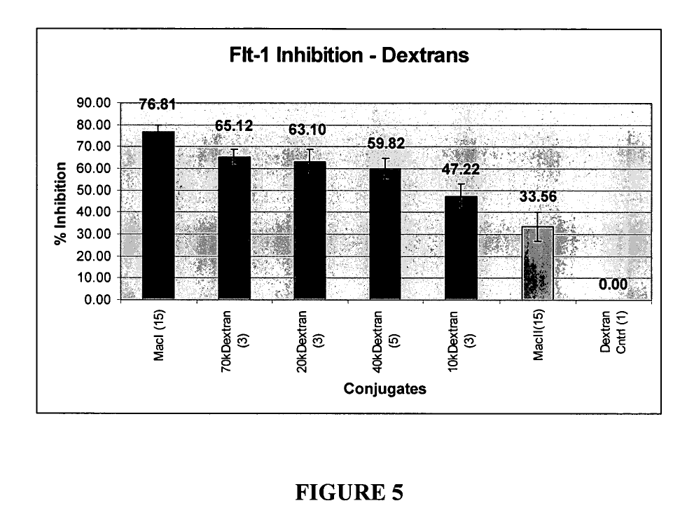

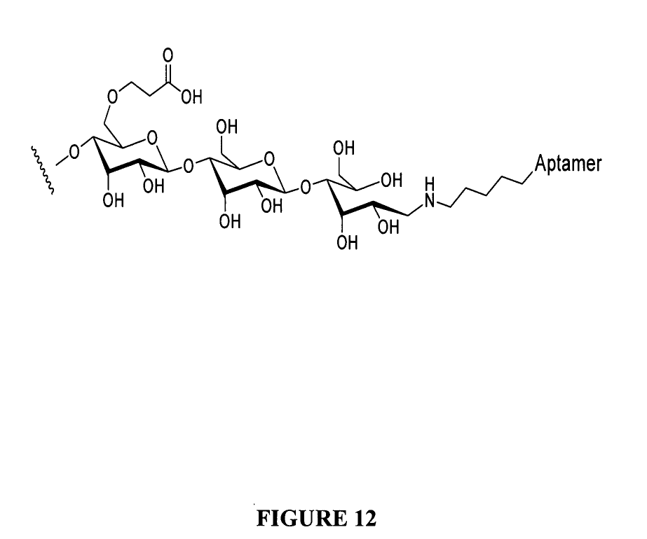

An example of the chemical structure of a dextran conjugated aptamer is shown in FIG. 12. The activity of dextran-VEGF aptamer conjugates was determined by a VEGFR-1 (Flt-1) inhibition assay. A graphical representation of the assay results are shown in FIG. 5. The assays shown in FIG. 4 also compared dextrans of various molecular weights. For example a conjugate having a 70 kDa dextran (70 KDextran) was compared to a conjugate having a 10 kDa dextran (10 KDextran). Significantly, non-conjugated dextran alone (control) did not inhibit binding of VEGF to Flt-1 indicating that these soluble, high molecular weight steric groups do not directly affect VEGF/Flt-1 binding, but act through the VEGF aptamer to which they are conjugated.

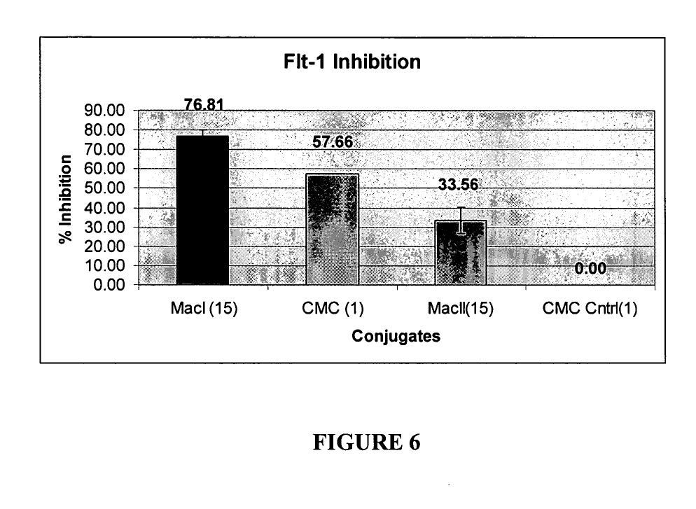

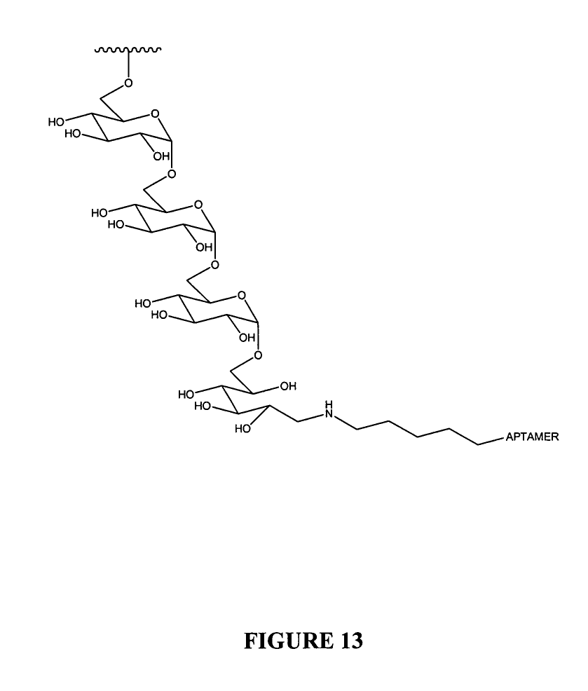

An example of the chemical structure of a CMC conjugated aptamer is shown in FIG. 13. The activity of CMC-VEGF aptamer conjugates was determined by a VEGFR-1 (Flt-1) inhibition assay. A graphical representation of the assay results are shown in FIG. 6. Significantly, non-conjugated CMC alone (control) did not inhibit binding of VEGF to Flt-1 indicating that these soluble, high molecular weight steric groups do not directly affect VEGF/Flt-1 binding, but act through the VEGF aptamer to which they are conjugated.

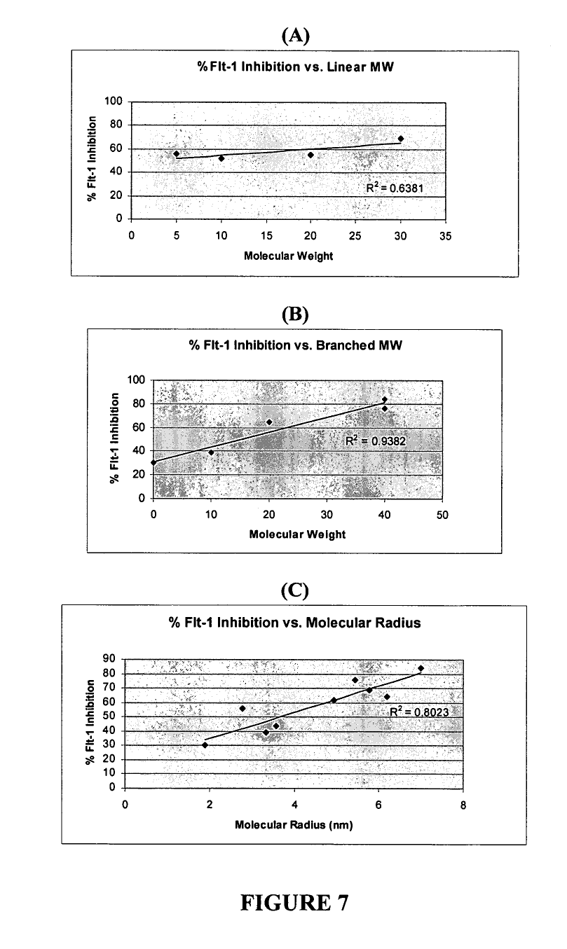

FIG. 7 shows the results of a VEGFR-1 (Flt-1) inhibition assay using various PEGylated VEGF aptamer conjugates having PEG moieties of various molecular weights and molecular radii (hydrodynamic volumes). The effectiveness of the sterically enhanced VEGF aptamer conjugates also correlated with the molecular weight of the soluble, high molecular weight steric group that was added. The effectiveness of the sterically enhanced VEGF aptamer conjugates also correlated with the molecular radius (hydrodynamic volume) of the soluble, high molecular weight steric group that was added.

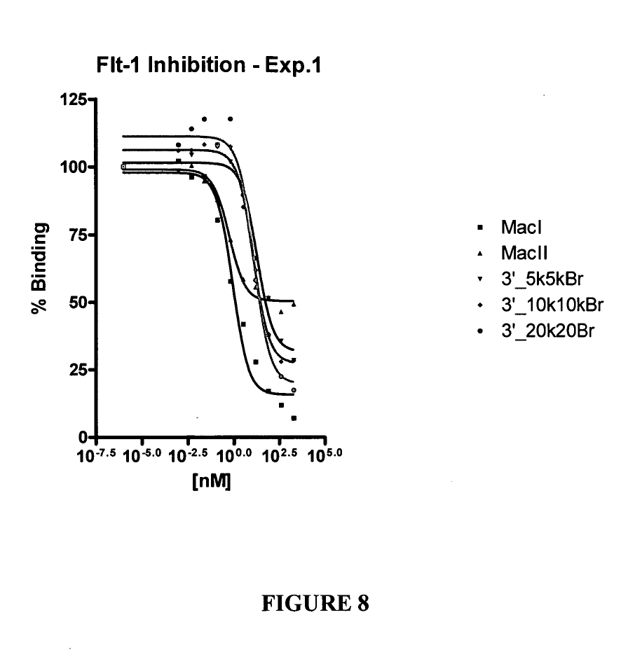

FIG. 8 shows the results of a VEGFR-1 (Flt-1) inhibition assay using various 3′-PEGylated VEGF aptamer conjugates. The results showed that conjugating PEG to the 3′-end of the VEGF aptamer was more effective in inhibiting VEGF binding than the non-enhanced VEGF aptamer. The results also showed that the soluble, high molecular weight steric groups may be placed at various locations on the aptamer.

The invention also provides a method of delivering a biologically active molecule to an eye comprising the steps of: a) attaching a charged molecule to the biologically active molecule forming a biologically active molecule charged conjugate; and b) delivering the biologically active molecule charged conjugate to the eye using iontophoresis.

The invention also relates to formulations useful for iontophoretic delivery of a biologically active molecule to an eye. The formulations comprise a biologically active molecule conjugated to a charged molecule. In one embodiment, the formulation comprises comprise a biologically active molecule conjugated to a charged molecule and a carrier suitable for iontophoretic delivery.

Any carrier suitable for iontophoretic delivery can be used in the present invention. Examples of suitable carriers include, but are not limited to, those that can be found in U.S. Pat. Nos. 6,154,671 6,319,240; 6,539,251; 6,579,276; 6,697,668; 6,728,573; 6,801,804 and 6,553,255, U.S. Patent Application Nos. 2004/0167459, 2004/0071761 and 2003/0065305, and published applications WO 2004/105864 and WO 2004/052252, the contents of each are incorporated herein by reference in their entirety.

In one aspect, the charged molecule is attached to the biologically active molecule by a hydrolytically stable bond.

In another aspect, the charged molecule comprises a charged polymer. In one embodiment, the charged polymer is a polyelectrolyte. In one embodiment the charged polymer is a high charge density polymer. In another embodiment the charged polymer is a high charge density polymer comprising a charge density ranging from about 1 to about 20 milliequivalents per gram (meq/g). In another embodiment the charged polymer is a high charge density polymer comprising a charge of at least 10 meq/g.

In one embodiment, the charged polymer is a cationic polymer. In a particular embodiment, the cationic polymer is chitosan.

In one embodiment, the charged polymer is an anionic polymer. In a particular embodiment, the anionic polymer is carboxymethyl cellulose (CMC).

In another aspect, the biologically active molecule is a nucleic acid. In one embodiment the nucleic acid is a ribonucleic acid (RNA), a deoxyribonucleic acid (DNA), an siRNA, an aptamer or an antisense oligonucleotide. A review of antisense oligonucleotides is provided by A. Mesmaeker et al. (“Antisense Oligonucleotides”, Acc. Chem. Res. 1995, 28, 366-374, which is hereby incorporated by reference in its entirety).

In one particular embodiment, the biologically active molecule is an aptamer. In another particular embodiment, the biologically active molecule is an anti-VEGF aptamer. In another particular embodiment, the biologically active molecule is the anti-VEGF aptaner, EYE-002, having the structure:

(SEQ ID NO: 1)

T_{d}-5′5′-C_{f}GₘGₘAᵣAᵣU_{f}C_{f}AₘGₘU_{f}GₘAₘAₘU_{f}GₘC_{f}U_{f}U_{f}AₘU_{f}
AₘC_{f}AₘU_{f}C_{f}C_{f}Gₘ 3′-3′-T_{d}

wherein “Gₘ” represents 2′-methoxyguanylic acid, “Aₘ” represents 2′-methoxyadenylic acid, “C_{f}” represents 2′-fluorocytidylic acid, “U_{f}” represents 2′-fluorouridylic acid, “Aᵣ” represents riboadenylic acid, and “T_{d}” represents deoxyribothymidylic acid. (See Adamis, A. P. et al., published application No. WO 2005/014814, which is hereby incorporated by reference in its entirety).

In a first example, the invention relates to a method of delivering a biologically active molecule to an eye comprising the steps of: a) attaching a charged molecule to the biologically active molecule by a hydrolytically stable bond, forming a biologically active molecule charged conjugate; and b) delivering the biologically active molecule charged conjugate to the eye using iontophoresis.

In a second example, the invention relates to a method of delivering nucleic acid to an eye comprising the steps of: a) attaching a non-nucleic acid polymer to a nucleic acid forming a nucleic acid charged conjugate; and b) delivering the nucleic acid charged conjugate to the eye using iontophoresis.

In a third example, the invention relates to a method of delivering an aptamer to an eye comprising the steps of: a) attaching an anionic high charge density polymer to the aptamer by a hydrolytically stable bond, forming an aptamer charged conjugate; and b) delivering the aptamer charged conjugate to the eye using iontophoresis.

In a fourth example, the invention relates to a method of delivering an anti-VEGF aptamer to an eye comprising the steps of: a) attaching a carboxymethyl cellulose or chitosan moiety to the anti-VEGF aptamer, forming an anti-VEGF aptamer charged conjugate; and b) delivering the anti-VEGF aptamer charged conjugate to the eye using iontophoresis.

Alternatively, the invention relates to a method of enhancing ocular iontophoresis. Iontophoretic delivery of a biologically active molecule that is conjugated to a high molecular weight neutral moiety is enhanced by substituting the high molecular weight neutral moiety with a charged molecule of comparable size. For example, a method of enhancing the iontophoretic delivery of a 5-100 kDa PEGylated aptamer comprises substituting the polyethylene glycol for a 5-100 kDa high charge density polymer such as carboxymethyl cellulose or chitosan.

The linkage between the biologically active agent-charged moiety conjugate should be stable in vitro and in vivo for extended periods of time. Further, the linkage should be stable upon application of an electric current, such as during iontophoretic delivery. A conjugate for use in iontophoresis should possess a physiologically stable bond which is stable upon application of an electric current. For example, for a biologically active agent-charged moiety conjugate intended for iontophoretic administration, the conjugate should maintain its integrity upon dissolution in an appropriate delivery vehicle, placement in the iontophoretic device, and upon application of electric current.

Any suitable current density may be used in the methods of the present invention. In one embodiment, the current density is adjustable between about 0.01 mA/cm² and about 5 mA/cm². In another embodiment, the current density is adjustable between about 0.1 mA/cm² and about 5 mA/cm². In another embodiment, the current density is adjustable between about 0.8 mA/cm² and about 5 mA/cm². In a one embodiment, the current is applied at a range from about 1 μA to about 1000 μA. In a preferred embodiment, the current is about 400 μA applied for about 4 minutes (a charge of 0.12 coulomb at density of 1.2 mA/cm²).

Any suitable electrical potential may be used in the methods of the present invention. In one embodiment, the current is delivered at a voltage ranging from about 1 V to about 75 V. In one embodiment, the current is delivered at a voltage ranging from about 1.5 V to about 9 V, and preferably ranging from about 2 V to about 8 V.

Any suitable iontophoretic device may be used in the present invention. Several ocular iontophoretic devices capable of delivering therapeutic levels of a biologically active molecule are known. A typical coulomb-controlled ocular iontophoretic device comprises 1) a reservoir of active product, for example, a biologically active molecule that can be applied to a patient's eye, 2) at least one active electrode arranged in the reservoir, 3) a passive electrode and 4) a current generator. Typically, one active electrode is a surface electrode arranged facing eye tissues lying at the periphery of the cornea. Such an iontophoretic device makes it possible to carry out ambulatory treatments.

Depending on the range of the surface area of the reservoir in contact with the eye, the iontophoretic device is optionally operated using a localized charge density system or diffuse charge density system.

Examples of iontophoretic devices and technologies useful in the present invention are provided herein:

Eyegate™, developed by Optis France, S. A., comprises two parts: a reusable micro-generator and a disposable ocular applicator. The disposable ocular applicator contains an inner ring that holds the drug and a conductive ring through which electric current is run to deliver the drug to the eye, particularly, the choroid and the retina. The reusable micro-generator is battery-powered with automatic control features, and is connected to a forehead patch that is used as a return electrode. The applicator, with its tubes, syringe (to inject the drug into the applicator) and lead (to connect to the micro-generator), is sterile, sealed into a blister, the whole being disposable. Iontophoretic devices and technologies relating to Eyegate™ are described, for example, in U.S. Pat. No. 6,154,671 and published applications WO 2004/105864, and WO 2004/052252, the contents of each are incorporated herein by reference in their entirety.

OcuPhor™, developed by Iomed, Incorporated, comprises a drug applicator, a dispersive electrode, and an electronic iontophoresis dose controller. The drug applicator is a small silicone shell that contains a silver-silver chloride ink conductive element; a hydrogel pad to absorb the drug formulation; and a small, flexible wire to connect the conductive element to the dose controller. The drug pad is hydrated with drug solution immediately prior to use, and the applicator is placed on the sclera of the eye under the lower eyelid. (see “OcuPhor™: The Future of Ocular Drug Delivery”, Fischer, G. A. et al., Drug Delivery Technology, 2002, 2(5), 50-52, the contents of which is incorporated herein by reference in its entirety). Iontophoretic devices relating OcuPhor™ are described, for example, in U.S. Pat. Nos. 6,319,240; 6,539,251; 6,579,276; 6,697,668; and 6,728,573, The contents of each are incorporated herein by reference in their entirety.

Visulex™, developed by Aciont, incorporated, consists of a user-friendly applicator, a dosing controller, and connecting wires. The device is designed for ophthalmic applications and contains software and algorithm controls and a multi-electrode monitoring system that together optimize safety. The applicator slips comfortably into the lower cul-de-sac, while conforming to the curvature of the eye. A fine, pliable wire connects the applicator to the current controller. The return electrode is positioned anywhere on the body to complete the electrical circuit Visulex™ system also comprises a membrane that increases drug transport efficiency over conventional iontophoretic systems by selective drug transport and flux enhancement. Excluding the transport of extraneous non-drug ions, maks drug ions the primary carrier of electrical current through scleral tissue. (see “Visulex™: Advancing Iontophoresis for Effective Noninvasive Back-of-the-Eye Therapeutics”, Hastings, M. S. et al., Drug Delivery Technology, 2004, 4(3), 53-57, the contents of which is incorporated herein by reference in its entirety.) Iontophoretic devices and technology relating to Visulex™ are described, for example, in U.S. Pat. Nos. 6,801,804 and 6,553,255, and U.S. Patent Application Nos. 2004/0167459, 2004/0071761 and 2003/0065305, the contents of each are incorporated herein by reference in their entirety.

Other ocular iontophoretic systems are described in the published application WO 03/0339622 by J. Ashook et al. (Ceramatec Inc.), U.S. Pat. No. 6,001,088 by M. S. Roberts et al. (University of Queensland), and U.S. Pat. No.6,442,423 by A. Domb et al. (Hadasit Medical Research Services and Development Limited and Yissum Research development company of the Hebrew university of Jerusalem). The contents of each are incorporated herein by reference in their entirety.

Literature reviews of ocular iontophoresis include “Ocular Iontophoresis”, Hill, J. M. et al., Drugs and the Pharmaceutical Sciences (1993), 58,331-54; and “The Role of Iontophoresis in Ocular Drug Delivery”, Sarraf, D. et al., Journal of Ocular Pharmacology (1994), 10(1), 69-81. The contents of each are incorporated herein by reference in their entirety.

The biologically active molecule may be attached to the charged molecule by any suitable means known in the art. The charge molecules can by attached to the biologically active molecule by means of an active functional group. Active functional groups suitable for reacting with biologically active molecules include, but are not limited to, carboxy, hydroxy, amino, sulfate, phosphate, keto and aldehyde groups.

In another aspect, the invention relates to the biologically active molecule charged conjugate compositions useful for iontophoretic delivery.

In one embodiment, the biologically active molecule charged conjugate has the formula:

(SEQ ID NO: 12)

CMC-NH-(CH₂)ₙ-C_{f}GₘGₘAᵣAᵣU_{f}C_{f}AₘGₘU_{f}GₘAₘAₘU_{f}GₘC_{f}U_{f}
U_{f}AₘU_{f}AₘC_{f}AₘU_{f}C_{f}C_{f}Gₘ.

In another embodiment, the biologically active molecule charged conjugate has the formula:

(SEQ ID NO: 13)

Chitosan-NH-(CH₂)ₙ-
C_{f}GₘGₘAᵣAᵣU_{f}C_{f}AₘGₘU_{f}GₘAₘAₘU_{f}GₘC_{f}U_{f}U_{f}AₘU_{f}AₘC_{f}AₘU_{f}C_{f}
C_{f}Gₘ.

### EXAMPLES

The following examples serve to illustrate certain useful embodiments and aspects of the present invention and are not to be construed as limiting the scope thereof. Alternative materials and methods can be utilized to obtain similar results.

### Example 1

Preparation of a 5′-PEG Conjugate of a VEGF Aptamer

The procedure is illustrated by the preparation of 40 kDa PEG/aptamer conjugate. A solution of 5′ amino aptamer (57 O.D.) was transferred to an Eppendorf tube and lyophilized to a solid. The residue was re-dissolved in 30 μL sodium borate buffer (0.1 M, pH 8.5). A solution of PEG NHS ester (1. 1 equiv., 11 mg in 30 μL acetonitrile) was added to the above aptamer solution. The resulting mixture was vortexed well and incubated at room temperature over night. The reaction was stopped by addition of water to a 2.5 mL volume. Analysis of the material by SEC HPLC indicates the aptamer (10.23 min.) was converted another species with much longer retention time (7.2 min., 75%), which belonged to the conjugate.

The mixture was desalted on a standard desalting column (Pharmacia PD-10 column). The desalted material (3.5 mL) was quantitated by UV (9.5 O.D./mL) and concentrated to a dry powder as the crude product. The solid was re-suspended in water (0.5 mL) and the resulting stock was stored in a −20° C. freezer until purification. Isolation of the conjugate was accomplished by injecting an aliquot of this solution (typically about 5 O.D.) using SEC HPLC. The eluted material corresponding to the conjugate was collected, concentrated on Speed-Vac and desalted to yield the purified conjugate. The product was finally analyzed by HPLC and MS to verify its identity.

### Example 2

Preparation of a 5′-Dextran Conjugate of a VEGF Aptamer

The procedure is illustrated by making a 40 kDa dextran/aptamer conjugate. An aliquot of amino aptamer (28.6 O.D.) was lyophilized to a dry powder and re-dissolved in 100 μL 0.1 M phosphate buffer (pH 7.0). To this solution were added 40 kDa dextran (4 equiv., 20 mg), and sodium cyanoborohydride (>10 equiv, 8 mg). The solution was vortexed to get all the materials dissolved and then incubated at 60° C. overnight. The solution was then taken up by 0.5 mL 0.1 M phosphate buffer (pH 7.0). HPLC (SEC) analysis indicated the material was a mixture of the aptamer (10.8 min) and the conjugate (9.6 min., broad peak, 35%). The broad peak indicates the dextran conjugate has a wide distribution of the conjugates of different sizes. The material was desalted by a PD-10 column and the desalted material was stored in a freezer (−20° C.) until purification.

Purification was performed on a SEC column (Showdex KW 803) by injecting an aliquot of the sample prepared above. The fractions corresponding to the conjugate (ambient temperature, 9.6 min) were collected. The pooled fractions were concentrated and then desalted on a NAP-10 column to yield the final purified material. The identity of the conjugate was verified by SEC HPLC (R. T. 9.6 min) with both UV and RI detections.

### Example 3

Preparation of a 5 ′-CMC Conjugate of a VEGF Aptamer

A procedure similar to that used in making dextran conjugates (See Example 2) was used to make the 5′-CMC conjugation of VEGF aptamer. A 5′-amino VEGF aptamer (28 O.D.) was lyophilized to a solid residue in an Eppendorf tube and dissolved in 0.1 M phosphate buffer (pH 7.0, 100 μL). To this was added 20 mg (3.2 equiv.) CMC. The molecular weight of the CMC was approximately 50 kDa. An additional aliquot of water (100 μL) was then added to solublize the CMC polymer, yielding a thick, viscous solution. Finally, sodium cyanoborohydride (8 mg) was added. After stirring overnight at 60° C., the reaction was stopped by diluting with water (about 2 mL) and dialyzing in water (3 times) to yield the crude conjugation material. SEC HPLC indicated the presence of the conjugated product (5.8 to 8.3 min.). Fractions corresponding to the conjugate were collected and desalted to yield the sample for functional testing. The conjugate appears as a very broad peak on IE HPLC, reflecting the fact that material is a polyanionic polymer.

### Example 4

Preparation of a 3 ′-PEG Conjugate of a VEGF Aptamer

A solution of 3′ amino aptamer (57 O.D.) was transferred to an Eppendorf tube and lyophilized to a solid. The residue was re-dissolved in 90 μL sodium borate buffer (0.1 M, pH 8.5). A solution of polyethylene glycol-N-hydroxysuccinimide (PEG-NHS) ester (1.1 equiv., 30 μL acetonitrile) was added to the above aptamer solution. The resulting mixture was vortexed well and incubated at room temperature over night. The reaction was stopped by addition of water to a 2.5 mL volume. Analysis of the material by size exclusion chromatography (SEC) HPLC indicates the aptamer was converted another species with much longer retention time, which belonged to the conjugate.

The mixture was desalted on a standard desalting column (Pharmacia PD-10 column). The desalted material (3.5 mL) was quantitated by UV and concentrated to a dry powder as the crude product. The solid was re-suspended in water (0.5 mL) and the resulting stock was stored in a −20° C. freezer until purification. Isolation of the conjugate was done by injecting an aliquot of this solution (typically about 5 O.D.) using SEC HPLC. The eluted material corresponding to the conjugate was collected, concentrated on Speed-Vac and desalted to yield the purified conjugate. The product was finally analyzed by HPLC and MS to verify its identity.

### Example 5

Conjugation of an Amine-Containing Aptamer to Bifunctional Linkers

1.30 micromoles of a 28mer oligonucleotide (SEQ ID NO: 8) containing a hexylamine linker attached to the 5′ terminus by a phosphodiester bond was dissolved in 200 μL of borate buffer (100 mM, pH 8.5), and a solution of the N-hydroxy succinimide ester-containing, bifunctional linker (8.0 micromoles) in 200 L of acetonitrile was added at room temperature. The resulting reaction mixture was shaken at room temperature for 18 h, then diluted to 3 mL with deionized water and spin dialyzed at 3520× g for 4 h against a 1 kDa membrane. The resulting concentrate was again diluted to 3 mL and spin dialyzed a second time. The resulting concentrate was then lyophilized and modification assessed by reverse phase HPLC chromatography (Hamilton PRP-1, C18) and MALDI-MS. Bifunctional linkers (6-maleimidocaproic acid NHS; Succinimidyl-2-(t-butoxycarbonylhydrazino)acetate; N-succinimidyl-3-(2-pyridyldithio)propionate) were purchased from Molecular Biosciences; Boulder, Colo. A general synthetic scheme representing the conjugation of a bifunctional linker to an aptamer is shown in FIG. 16.

### Example 6

Aptamer Conjugation to BSA

Conjugation of bovine serum albumin (BSA) to an aptamer (SEQ ID NO: 8) that has been modified with a thiol-reactive bifunctional linker was performed in phosphate buffer (0.1 M Na2PO3, 0.15M NaCl, pH 7.7). BSA solution (692 μL, 40 mg/mL) was added to a solution of the aptamer conjugate (300 nM in 300 μL) and shaken at room temperature for 4 h at ambient temperature. The reaction mixture was analyzed and was subject to purification on reverse phase HPLC (Waters Deltapak, C18) without further processing. BSA was purchased from Sigma-Aldrich.

### Example 7

Aptamer Conjugation to a Dendron

A solution of dendrimer (G6, cystamine core, NHAc surface; commercially available from Sigma-Aldrich) was dissolved in methanol (2.1 mg in 50 μL) then treated with tris-carboxyethylphosphine (50 mg) in 50 μL of a phosphate buffer (0.1 M Na₂PO₃, 0.15 M NaCl, pH 7.7) and shaken at 30 min at ambient temperature. A solution of aptamer (SEQ ID NO: 8, modified with a thiol-reactive bifunctional linker (3.0 mg)) was prepared by adding the aptamer to 100 μL of a phosphate buffer (0.1 M Na₂PO₃, 0.15 M NaCl, pH 7.7). The aptamer solution was then added to the dendrimer solution and the resulting solution stirred for 1 h at room temperature. The solution was lyophilized and the product characterized and purified by size exclusion chromatography (Shodex KW-803 & KW-804 in sequence).

### Example 8

Sterically Enhanced ICAM, PDGF and VEGF Antagonist Aptamers

The ability of sterically-enhanced VEGF aptamers to inhibit the binding of VEGF to KDR/FLK-1 (VEGF-R2), FLT-1 (VEGF-R1) and the VEGF co-receptor Neuropilin is assessed as follows. Inhibition of binding by the sterically enhanced aptamers is compared to inhibition by non-enhanced aptamers.

The ability of sterically enhanced ICAM-1 aptamers to inhibit binding to LFA-1 is also examined using similar procedures. The ability of sterically enhanced PDGF aptamers to inhibit the binding of PDGF to PDGF receptor-beta (PDGFR-β) is also examined using similar procedures.

### Example 9

Receptor Plate Coating

For each set of binding experiment, one row (12 wells) of a 96-well Isoplate Plate is used. Each of the 12 wells is first coated with 2 picomole (300 nanograms (ng)) of anti-human IgG1 Fc fragment-specific antibody in 100 microliter (μL) of PBS at 4° C. overnight. The next day, further protein binding in each well is blocked by washing with 300 μL of Super Block blocking buffer at room temperature for 3 times, 5 minutes each. Each well is then washed with 300 μL of binding buffer (PBS with 1 mM calcium chloride, 1 mM magnesium chloride, 0.01% HSA, PH 7.4) at room temperature twice. For KDR/Fc, 0.25 picomole (85 ng) of the chimeric receptor in 100 μL of binding buffer is added into the first 11 wells, whereas the twelfth well receive 0.5 picomole (118 ng) of human ICAM-1/Fc chimera protein as the background control well. For Flt-1/Fc, 0.125 picomole (30.8 ng) of the chimeric receptor in 100 μL of binding buffer each is added into the first 11 wells, whereas the background control well (#12) receive 0.5 picomole (118 ng) of human ICAM-1/Fc chimera protein. For neuropilin-1/Fc, 0.2 picomole (48 ng) of the chimeric receptor in 100 μl of binding buffer is added to all 12 wells. The chimeric receptors and human ICAM-1/Fc protein are captured onto the well by binding to the immobilized anti-human IgG₁ Fc fragment-specific antibody in each well at room temperature for 2 to 3 hour. Each well is washed with 300 μL of binding buffer at room temperature to remove the free chimeric receptors and human ICAM-1/Fc protein.

### Example 10

Preparation of ¹²⁵I-VEGF₁₆₅-Pegaptanib Binding Mix

A set of 10 five-fold dilutions of the Pegaptanib (tube #1 to #10) ranging from 1 μM (or 2 μM) to 0.512 picomolar (pM) (or 1.024 pM) are each mixed with about 0.01 μCi of ¹²⁵I-VEGF₁₆₅ in binding buffer (PBS with 1 mM calcium chloride, 1 mM Magnesium Chloride, 0.01% HSA, pH 7.4) in non-stick 1.5 mL microfuge tubes, in a total 100 μL final volume each. For tube #11 and #12, only 0.01 μCi of ¹²⁵I-VEGF₁₆₅ are added without any Pegaptanib and they are the positive and background controls, respectively. All 12 tubes are incubated at 37° C. (for KDR and Flt-1) or at room temperature (for neuropilin-1) for 15 to 20 min to allow the binding of Pegaptanib to VEGF to reach equilibrium. The 100 μL binding mix from each tube is then applied to the corresponding well on the receptor-coated Isoplate. The plate is incubated at 37° C. (for KDR and Flt-1) or at room temperature (for neuropilin-1) for 2 to 3 hours to allow equilibrium binding to occur. The plate is washed 4 times with 300 μL/well of binding buffer with (for KDR and neuropilin-1) or without (for Flt-1) 0.05% Tween 20, at room temperature. The plate is air dried for about 10 min, and about 200 μl of scintillation fluid is added to each well. The radioactivity of each well is determined by scintillation counting.

For experimental negative control, polyethylene glycol 40,000 MW (40 kDa PEG) is used at identical molar concentration to replace the Pegaptanib in the binding assay, following all the steps described above for Pegaptanib.

### Example 11

Determining Effective Concentration for 50% Inhibition (IC₅₀) of VEGF Receptor Binding

The ¹²⁵I-VEGF₁₆₅:receptor binding ratios in the wells are calculated as: number of counts retained on the wells (#1 to #11) minus the background (well #12) divided by the maximum binding (positive control, well #11) minus the background (well #12). The resulting binding ratios at different pegaptanib concentrations are analyzed by using nonlinear regression with the GraphPad PRISM program (one site competition), and the resulting curve is used to determine the half-maximum inhibition (IC₅₀) of pegaptanib in inhibiting the receptor binding to VEGF₁₆₅. Data from the experimental negative control using PEG are analyzed by the same method.

### Example 12

Comparative Inhibition of VEGF-R1 (Flt-1)

The ability of sterically enhanced VEGF aptamer conjugates to inhibit VEGF binding to VEGF-R1 (Flt-1) was compared to that of non-sterically enhanced VEGF aptamer conjugates. The results are shown in FIGS. 4, 5, 6 7 and 8. The results indicate that sterically enhanced VEGF aptamer conjugates such as Pegaptanib (EYE-001, MacI, the structure of which is shown in FIG. 1) are much more effective in inhibiting VEGF binding than are non-enhanced VEGF aptamers such as EYE-002 (MacII) (the structure of which is shown in FIG. 2). Furthermore, the effectiveness of the sterically enhanced VEGF aptamer conjugates correlated with the molecular weight of the soluble, high molecular weight steric group that was added (compare 20K/20K Branched to 5K/5K Branched, and 30K Linear to 10K Linear). The effectiveness of the sterically enhanced VEGF aptamer conjugates also correlated with the molecular radius (hydrodynamic volume) of the soluble, high molecular weight steric group that was added. Significantly, neither non-conjugated PEG, Dextran or CMC alone affected binding of VEGF to Flt-1 indicating that these soluble, high molecular weight steric groups do not directly affect VEGF/Flt-1 binding, but act through the VEGF aptamer to which they are conjugated.

Results showing that conjugating PEG to the 3′-end of the VEGF aptamer was more effective in inhibiting VEGF binding than the non-enhanced VEGF aptamer indicated that the soluble, high molecular weight steric groups may be placed at various locations on the aptamer.

### Example 13

Design of an ICAM-1 Sterically Enhanced Aptamer Antagonist

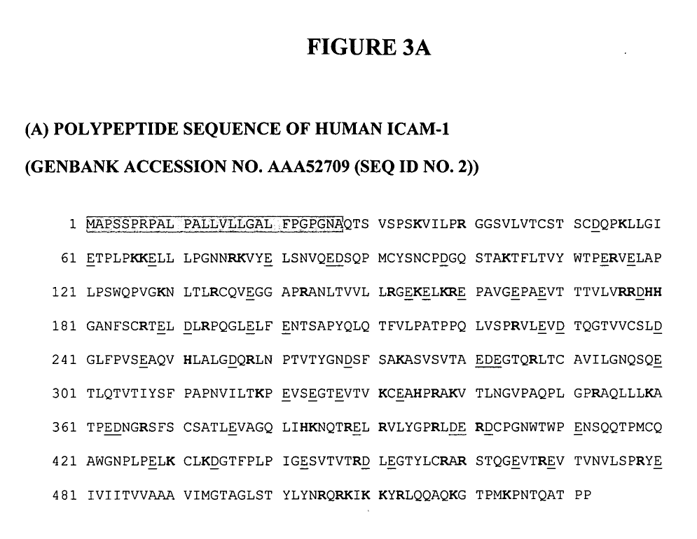

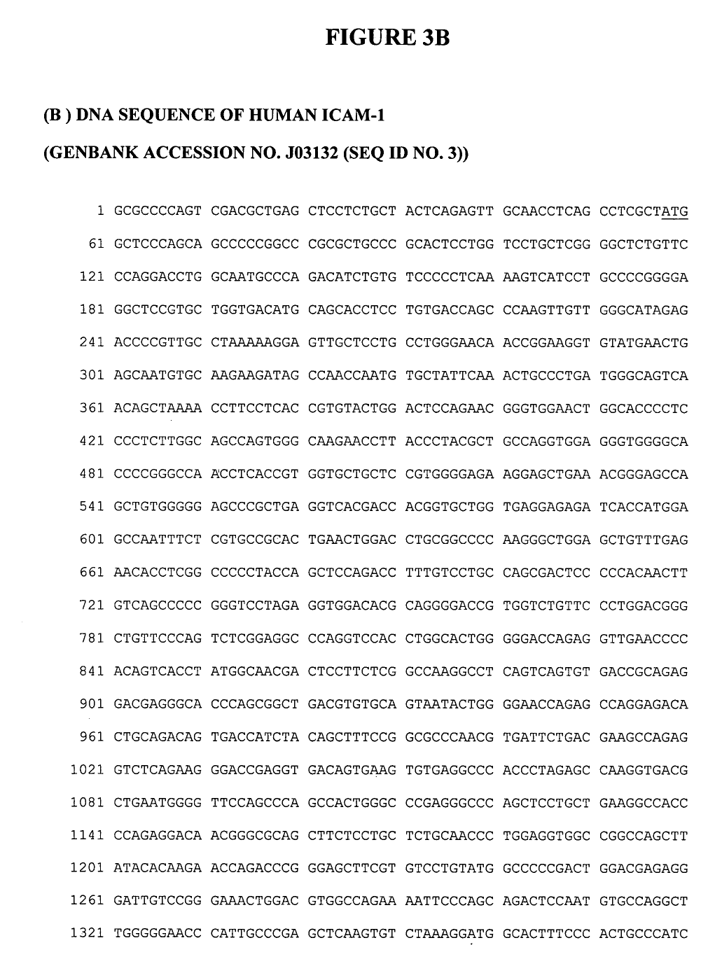

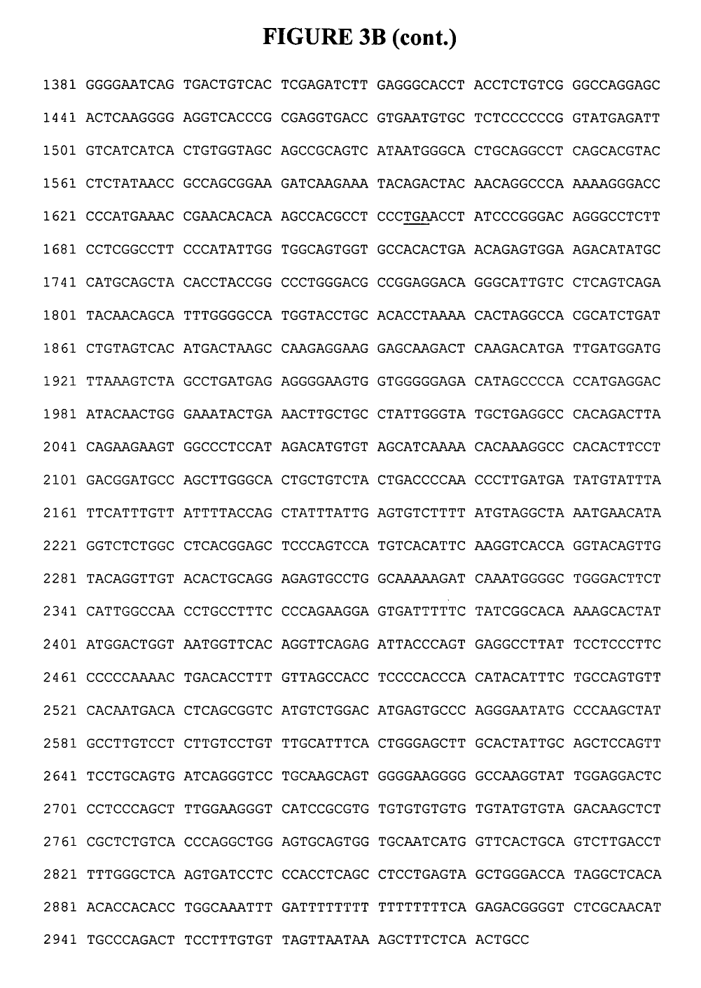

ICAM-1 is an intercellular adhesion molecule. It is a single-membrane spanning protein, with 5 Ig-like extracellular domains, located primarily on endothelial cells and certain blood cell types. It has two well recognized receptors, LFA-1 and Mac-1, which belong to the integrin family of adhesion receptors. Domain 1 of ICAM-1 is the LFA-1 interaction domain and is the focus of most drug development approaches. However this domain of ICAM-1 is highly acidic (pI of 4.5-5) and, accordingly, it is difficult to select for, or otherwise design, aptamer sequences that are capable of directly blocking ICAM-1/LFA-1 interaction by binding directly to it. In contrast, the adjacent domain 2 of ICAM-1 is highly basic (pI 8-9.5) and, accordingly, is a more amenable aptamer binding region (see FIG. 3(A) and Figure 11, left).

Accordingly, the basic domain 2 of ICAM-1 is used to select aptamer sequences that bind with high affinity to this region of ICAM-1. High molecular weight, soluble steric groups are then added to the aptamer to effect steric inhibition of an interaction between LFA-1 and the adjacent domain 1 of ICAM-1 (FIG. 11, right). The aptamer serves as a foothold or anchor, while the high molecular weight steric group is attached on an end of the aptamer that would cause it to block the acidic LFA-1-binding domain of ICAM-1.

### Example 14

Iontophoresis of an Anti-VEGF Aptamer Conjugated to Carboxymethyl Cellulose

Coulomb-controlled Iontophoresis (CCI) system Iontophoresis can be performed using the drug delivery device designed by OPTIS France (see U.S. Pat. No. 6,154,671, and WO 02/083184, by Optis, which are each incorporated herein by reference in their entirety). A container, in the form of an ocular cup, is designed to allow transcorneoscleral iontophoresis. A platinum electrode is placed at the bottom of the container and two silicone tubes are settled laterally. An iontophoretic formulation comprising an anti-VEGF aptamer conjugated to carboxymethyl cellulose is added to the container. One tube is used to infuse saline buffer and the other is used to aspirate bubbles. The CCI electronic unit can deliver up to 2,500 microamperes (μA) for 600 seconds. An audio-visual alarm indicates each disruption in the electric circuit ensuring a calibrated and controlled delivery of the product. To proceed with the iontophoresis treatment, the CCI ocular cup, containing the iontophoretic formulation comprising an anti-VEGF aptamer conjugated to carboxymethyl cellulose, is placed on the eye and the other electrode is maintained in contact with the subject.

### Example 15

Iontophoresis of an Anti-VEGF Aptamer Conjugated to Carboxymethyl Cellulose

Iontophoretic delivery of a anti-VEGF aptamer conjugated to carboxymethyl cellulose is performed using an ocular rabbit ophthalmic applicator (IOMED Inc., Salt Lake City, Utah) composed of an 180 μL silicone receptacle shell backed with silver chloride-coated silver foil current distribution component, a connector lead wire, and a single layer of hydrogel-impregnated polyvinyl acetal matrix to which the anti-VEGF aptamer conjugate is administered. The contact surface area of the applicator is 0.54 cm². The applicator is placed over the sclera in the right eyes of New Zealand white rabbits (3-3.5 kg) in the superior cul-de-sac at the limbus with the front edge 1-2 mm distal from the corneoscleral junction. Direct current anodal iontophoresis is performed with each applicator at 2, 3, and 4 mA for 20 min using an Phoresor II™ PM 700 (IOMED Inc., Salt Lake City, Utah) power supply. Passive iontophoresis (0 mA for 20 min) is used as a control.

All patents, patent applications, and published references cited herein are hereby incorporated by reference in their entirety.

Equivalents

Those skilled in the art will recognize, or be able to ascertain, using no more than routine experimentation, numerous equivalents to the specific embodiments described specifically herein. Such equivalents are intended to be encompassed in the scope of the following claims.

1. A method of inhibiting activity of a site separate from an aptamer binding site on a ligand comprising:
joining an aptamer to a soluble, high molecular weight steric group, wherein the soluble, high molecular weight steric group inhibits the activity of the site separate from to the aptamer binding site on the ligand.

2. The method of claim 1, wherein the soluble high molecular weight steric group is selected from the group consisting of a polysaccharide, a glycosaminoglycan, a hyaluronan, an alginate, a polyester, a high molecular weight polyoxyalkylene ether, a polyalkylene glycol, a polyamide, a polyurethane, a polysiloxane, a polyacrylate, a polyol, a polyvinylpyrrolidone, a polyvinyl alcohol, a polyanhydride, a dendron, a dextran, a cellulose, a cellulose derivative, a carboxymethyl cellulose, a carboxymethyl dextran, a chitosan, a polyadlehyde, a polylactide-co-glycolide, and a polyether.

3. The method of claim 1, wherein the soluble, high molecular weight steric group is dextran or a derivative thereof.

4. The method of claim 1, wherein the soluble, high molecular weight steric group is polyethylene glycol.

5. The method of claim 1, wherein the soluble high molecular weight steric group is a polymeric composition having a molecular weight of 800 Da to 3,000,000 Da.

6. The method of claim 1, wherein the soluble high molecular weight steric group is a polymeric composition having a molecular weight of 20 kDa to 1000 kDa.

7. The method of claim 1, wherein the soluble high molecular weight steric group is a polymeric composition having a molecular weight of 20 to 100 kDa.

8. The method of claim 1, wherein the soluble high molecular weight steric group is a polymeric composition having a molecular weight of about 20 kDa.

9. The method of claim 1, wherein the soluble high molecular weight steric group is a polymeric composition having a molecular weight of about 40 kDa.

10. The method of claim 1, wherein the soluble high molecular weight steric group is a polymeric composition having a molecular weight of about 80 kDa.

11. The method of claim 1, wherein the soluble high molecular weight steric group is a polymeric composition having a hydrodynamic radius of 1 to 10 nm.

12. The method of claim 1, wherein the soluble high molecular weight steric group is a polymeric composition having a hydrodynamic radius of about 4 nm.

13. The method of claim 1, wherein the soluble high molecular weight steric group is a polymeric composition having a hydrodynamic radius of about 6 nm.

14. The method of claim 1, wherein the aptamer is directed to a ligand or its receptor selected from the group consisting of a growth factor, vascular endothelial growth factor (VEGF), TGF, TGFβ, PDGF, IGF, FGF, cytokine, lymphokine, hematopoietic factor, M-CSR, GM-CSF, TNF, interleukin, IL-1, IL-2, IL-3, IL-4, IL-5, IL-6, IL-7, IL-8, IL-9, IL-10, IL-11, IL-12, IL-1 3, IL-14, IL-15, IL-16, IL-17, IL18, IFN, TNF0, TNF1, TNF2, G-CSF, Meg-CSF, GM-CSF, thrombopoietin, stem cell factor, erythropoietin, hepatocyte growth factor/NK1, angiogenic factor, angiopoietin, Ang-1, Ang-2, Ang-4, Ang-Y, human angiopoietin-like polypeptide, angiogenin, morphogenic protein-1, bone morphogenic protein receptor, bone morphogenic protein receptor IA, bone morphogenic protein receptor IB, neurotrophic factor, chemotactic factor, CD proteins, CD3, CD4, CD8, CD19, CD20, erythropoietin, osteoinductive factors, immunotoxin, bone morphogenetic protein (BMP), interferon, interferon-alpha, interferon-beta, interferon-gamma, colony stimulating factor (CSF), M-CSF, GM-CSF, G-CSF, superoxide dismutase, T-cell receptor; surface membrane protein, decay accelerating factor, viral antigen, portion of the AIDS envelope, transport protein, homing receptor, addressin, regulatory protein, integrin, CD11a, CD11b, CD11c, CD18, ICAM, VLA-4, VCAM, tumor associated antigen, HER2, HER3, and HER4 receptor; or fragments or variants thereof.

15. The method of claim 1, wherein the aptamer is directed to VEGF-A.

16. The method of claim 1, wherein the aptamer is directed to VEGF-165.

17. The method of claim 1, wherein the aptamer is directed to a VEGF ligand selected from the group consisting of VEGF-B, VEGF-C, VEGF-D, and VEGF-E.

18. The method of claim 1, wherein the aptamer is directed to a VEGF receptor.

19. The method of claim 18, wherein the VEGF receptor is Flk-1I/KDR (VEGFR-2).

20. The method of claim 18, wherein the VEGF receptor is Flt-1 (VEGFR-1).

21. The method of claim 18, wherein the VEGF receptor is Flt-4 (VEGFR-3).

22. The method of claim 1, wherein the aptamer is directed to a VEGF co-receptor.

23. The method of claim 22, wherein the VEGF co-receptor is neuropilin-1 or neuropilin-2.

24. The method of claim 22, wherein the VEGF co-receptor is αVβ3 integrin or VE-cadherin.

25. The method of claim 1, wherein the aptamer is directed to ICAM-1.

26. The method of claim 1, wherein the aptamer is directed to LFA-1.

27. The method of claim 1, wherein the aptamer comprises the sequence:
(SEQ ID NO: 8)
C_{f}GₘGₘAᵣAᵣU_{f}C_{f}AₘGₘU_{f}GₘAₘAₘU_{f}GₘC_{f}U_{f}U_{f}AₘU_{f}AₘC_{f}AₘU_{f}C_{f}
C_{f}Gₘ.

28. The method of claim 1, wherein the site separate from an aptamer binding site is a site distal to the aptamer binding site.

29. The method of claim 1, wherein the site separate from an aptamer binding site is a site proximal to the aptamer binding site.

30. The method of claim 1, wherein the site separate from an aptamer binding site is negatively charged or neutral and the aptamer binding site is positively charged.

31. A method of increasing the receptor antagonist range of a ligand-binding aptamer, wherein the ligand binds to multiple receptors and wherein the ligand-binding aptamer fails to effectively antagonize the ligand-dependent activation of at least one of the multiple receptors comprising:
joining the aptamer to a soluble, high molecular weight steric group, wherein the aptamer, when joined to the soluble, high molecular weight steric group, effectively antagonizes the ligand-dependent activation of the at least one receptor that is not otherwise effectively antagonized by the aptamer alone.

32. The method of claim 31, wherein the soluble high molecular weight steric group is selected from the group consisting of a polysaccharide, a glycosaminoglycan, a hyaluronan, an alginate, a polyester, a high molecular weight polyoxyalkylene ether, a polyalkylene glycol, a polyamide, a polyurethane, a polysiloxane, a polyacrylate, a polyol, a polyvinylpyrrolidone, a polyvinyl alcohol, a polyanhydride, a dendron, a dextran, a cellulose, a cellulose derivative, a carboxymethyl cellulose, a carboxymethyl dextran, a chitosan, a polyadlehyde and a polyether.

33. The method of claim 31, wherein the soluble, high molecular weight steric group is dextran or a derivative thereof.

34. The method of claim 31, wherein the soluble, high molecular weight steric group is polyethylene glycol.

35. The method of claim 31, wherein the soluble high molecular weight steric group is a polymeric composition having a molecular weight of 800 Da to 3,000,000 Da.

36. The method of claim 31, wherein the soluble high molecular weight steric group is a polymeric composition having a molecular weight of 20 kDa to 1000 kDa.

37. The method of claim 31, wherein the soluble high molecular weight steric group is a polymeric composition having a molecular weight of 20 to 100 kDa.

38. The method of claim 31, wherein the soluble high molecular weight steric group is a polymeric composition having a molecular weight of about 20 kDa.

39. The method of claim 31, wherein the soluble high molecular weight steric group is a polymeric composition having a molecular weight of about 40 kDa.

40. The method of claim 31, wherein the soluble high molecular weight steric group is a polymeric composition having a molecular weight of about 80 kDa.

41. The method of claim 31, wherein the soluble high molecular weight steric group is a polymeric composition having a hydrodynamic radius of 1 to 10 nm.

42. The method of claim 31, wherein the soluble high molecular weight steric group is a polymeric composition having a hydrodynamic radius of about 4 nm.

43. The method of claim 31, wherein the soluble high molecular weight steric group is a polymeric composition having a hydrodynamic radius of about 6 nm.

44. The method of claim 31, wherein the aptamer is directed to a ligand or its receptor selected from the group consisting of a growth factor, vascular endothelial growth factor (VEGF), TGF, TGFβ, PDGF, IGF, FGF, cytokine, lymphokine, hematopoietic factor, M-CSR, GM-CSF, TNF, interleukin, IL-1, IL-2, IL-3, IL-4, IL-5, IL-6, IL-7, IL-8, IL-9, IL-10, IL-11, IL-12, IL-13, IL-14, IL-15, IL-16, IL-17, IL18, IFN, TNF0, TNF1, TNF2, G-CSF, Meg-CSF, GM-CSF, thrombopoietin, stem cell factor, erythropoietin, hepatocyte growth factor/NK1, angiogenic factor, angiopoietin, Ang-1, Ang-2, Ang-4, Ang-Y, human angiopoietin-like polypeptide, angiogenin, morphogenic protein-1, bone morphogenic protein receptor, bone morphogenic protein receptor IA, bone morphogenic protein receptor IB, neurotrophic factor, chemotactic factor, CD proteins, CD3, CD4, CD8, CD19, CD20, erythropoietin, osteoinductive factors, immunotoxin, bone morphogenetic protein (BMP), interferon, interferon-alpha, interferon-beta, interferon-gamma, colony stimulating factor (CSF), M-CSF, GM-CSF, G-CSF, superoxide dismutase, T-cell receptor; surface membrane protein, decay accelerating factor, viral antigen, portion of the AIDS envelope, transport protein, homing receptor, addressin, regulatory protein, integrin, CD11a, CD11b, CD11c, CD18, ICAM, VLA-4, VCAM, tumor associated antigen, HER2, HER3, and HER4 receptor; or fragments or variants thereof.

45. The method of claim 31, wherein the aptamer is directed to VEGF-A.

46. The method of claim 31, wherein the aptamer is directed to VEGF-165.

47. The method of claim 31, wherein the aptamer is directed to a VEGF ligand selected from the group consisting of VEGF-B, VEGF-C, VEGF-D, and VEGF-E.

48. The method of claim 31, wherein the aptamer is directed to a VEGF receptor.

49. The method of claim 48, wherein the VEGF receptor is Flk-1/KDR (VEGFR-2).

50. The method of claim 48, wherein the VEGF receptor is Flt-1 (VEGFR-1).

51. The method of claim 48, wherein the VEGF receptor is Flt-4 (VEGFR-3).

52. The method of claim 31, wherein the aptamer is directed to a VEGF co-receptor.

53. The method of claim 52, wherein the VEGF co-receptor is neuropilin-1 or neuropilin-2.

54. The method of claim 52, wherein the VEGF co-receptor is αVβ3 integrin or VE-cadherin.

55. The method of claim 31, wherein the aptamer is directed to ICAM-1.

56. The method of claim 31, wherein the aptamer is directed to LFA-1.

57. The method of claim 31, wherein the aptamer comprises the sequence:
(SEQ ID NO: 8)
C_{f}GₘGₘAᵣAᵣU_{f}C_{f}AₘGₘU_{f}GₘAₘAₘU_{f}GₘC_{f}U_{f}U_{f}AₘU_{f}AₘC_{f}AₘU_{f}C_{f}
C_{f}Gₘ.

58. A method of increasing the ligand antagonist range of a receptor-binding aptamer, wherein the receptor binds to multiple ligands and wherein the receptor-binding aptamer fails to effectively antagonize the ligand-dependent activation of at least one of the multiple ligands comprising:
joining the aptamer to a soluble, high molecular weight steric group, wherein the aptamer, when joined to the soluble, high molecular weight steric group, effectively antagonizes the ligand-dependent activation of the at least one ligand that is not otherwise effectively antagonized by the aptamer alone.

59. A method of increasing the receptor antagonist range of a VEGF aptamer comprising:
providing a VEGF aptamer that binds to VEGF, but that fails to effectively antagonize VEGF-dependent activation of at least one VEGF receptor, joining the VEGF aptamer to a soluble, high molecular weight steric group so that the resulting VEGF aptamer conjugate effectively antagonizes VEGF-dependent activation of the at least one VEGF receptor, thereby increasing the receptor antagonist range of the VEGF aptamer.

60. A method of increasing the ligand antagonist range of a VEGFR aptamer comprising:
providing a VEGFR aptamer that binds to a VEGFR, but that fails to effectively antagonizes ligand-dependent activation by at least one VEGF ligand, joining the VEGFR aptamer to a soluble, high molecular weight steric group so that the resulting VEGFR aptamer conjugate effectively antagonizes VEGFR-dependent activation by the at least one VEGF ligand, thereby increasing the ligand antagonist range of the VEGFR aptamer.

61. A method of increasing an antagonist property of an aptamer that targets a protein that interacts with a second protein comprising:
joining the aptamer to a soluble, high molecular weight steric group, wherein the soluble, high molecular weight steric group increases the ability of the aptamer to disrupt the interaction of the protein with the second protein and thereby increases an antagonist property of the aptamer.

62. A method for inhibiting binding of a ligand to a receptor, wherein the receptor binding site of the ligand is negatively charged and the aptamer binding site on the ligand is positively charged, comprising the step of:
joining an aptamer to a soluble, high molecular weight steric group forming an aptamer conjugate;
wherein the aptamer binds to the positively charged aptamer binding site on the ligand and the soluble, high molecular weight steric group inhibits the binding of the receptor to the negatively charged receptor binding site of the ligand.

63. The method of claim 62, wherein the receptor is an intercellular adhesion molecule (ICAM).

64. The method of claim 62, wherein the receptor is ICAM-1.

65. A method of identifying an aptamer conjugate having a stronger antagonist effect on a target than the corresponding non-conjugated aptamer, wherein the target is a ligand or a receptor of the ligand, comprising:
providing multiple aptamer conjugates that are, independently, joined to a soluble, high molecular weight steric group at the 5′- and, the 3′-end and, optionally, at one or more non 5′-terminal or 3′-terminal positions of the aptamer;
contacting, independently, each of said aptamer conjugates with the ligand and the receptor of the ligand and detecting the amount of ligand/receptor binding or ligand-dependent receptor activation; and
selecting the aptamer conjugate with the greatest ability to inhibit ligand/receptor binding or ligand-dependent receptor activation, thereby identifying an aptamer conjugate having a stronger antagonist effect on a ligand/receptor target than the corresponding non-conjugated aptamer.

66. A method of identifying an aptamer conjugate having an enhanced antagonist effect on a target, wherein the target is a ligand or a receptor of the ligand, the method comprising:
providing multiple aptamer conjugates that are, independently, joined to a soluble, high molecular weight steric group at the 5′-end, the 3′-end and, at one or more non 5′-terminal or 3′-terminal positions of the aptamer, wherein the soluble, high molecular weight steric group has a molecular weight of 20 to 100 kDa and is selected from the group consisting of a polysaccharide, a glycosaminoglycan, a hyaluronan, an alginate, a polyester, a high molecular weight polyoxyalkylene ether, a polyamide, a polyurethane, a polysiloxane, a polyacrylate, a polyol, a polyvinylpyrrolidone, a polyvinyl alcohol, a polyanhydride, a carboxymethyl cellulose, a cellulose derivative, a Chitosan, a polyaldehyde, and a polyether;
contacting, independently, each of said aptamer conjugates with the ligand and the receptor of the ligand;
detecting the amount of ligand/receptor binding or ligand-dependent receptor activation; and
selecting the aptamer conjugate with the greatest ability to inhibit ligand/receptor binding or ligand-dependent receptor activation,
wherein the aptamer conjugate has a stronger antagonist effect on a ligand/receptor target than the corresponding non-conjugated aptamer.

67. A compound comprising an aptamer conjugated to a high molecular weight steric group, wherein the aptamer is an anti-VEGF aptamer and the high molecular weight steric group is dextran, CMC, BSA, PLGA or a dendron.

68. The compound of claim 67 wherein the aptamer comprises the sequence
(SEQ ID NO: 8)
C_{f}GₘGₘAᵣAᵣU_{f}C_{f}AₘGₘU_{f}GₘAₘAₘU_{f}GₘC_{f}U_{f}U_{f}AₘU_{f}AₘC_{f}AₘU_{f}
C_{f}C_{f}Gₘ.
